SEQUENCE LISTING (91 sequences)

SEQ ID NO: 1 - protein, 230 aa, Homo sapiens
  REGION 1..230: note = human IgG1 isotype constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 276 aa, Homo sapiens
  REGION 1..276: note = human IgG2 isotype constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 3 - protein, 300 aa, Homo sapiens
  REGION 1..300: note = human IgG3 isotype constant region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQG

SEQ ID NO: 4 - protein, 227 aa, Homo sapiens
  REGION 1..227: note = human IgG4 isotype constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 118 aa, synthetic construct
  REGION 1..118: note = Synthetic polypeptide
EVQLQQSGAELVRSGASVKLSCTASGFNIKDYYLHWVKQRPEQGLEWIGWIDPDNGDTEFAPKFQGKATMTADTSSNTAYLQLSSLTSEDTAVYYCNAAYGSSSYPMDYWGQGTSVTV

SEQ ID NO: 6 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDQGDTEYAQKFQDRVTITRDRSMSTAYMELSSLRSEDTAMYYCNAAYGSSSYPMDYWGQGTTVTV

SEQ ID NO: 7 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 8 - protein, 118 aa, synthetic construct
  REGION 1..118: note = Synthetic polypeptide
EVQLVQSGAEVKKTGSSVKVSCKASGFNIKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 9 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDNGDTEYAQKFQGRVTMTADTSSNTAYMELSSLRSEDTAMYYCNAAYGSSSYPMDYWGQGTTVTV

SEQ ID NO: 10 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDNGDTEYAQKFQGRVTMTEDTSTDTAYMELSSLRSEDTAMYYCNAAYGSSSYPMDYWGQGTTVTV

SEQ ID NO: 11 - protein, 118 aa, synthetic construct; identical to SEQ ID NO: 6
  REGION 1..118: note = Synthetic polypeptide

SEQ ID NO: 12 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDYGDTEYAQKFQDRVTITRDRSMSTAYMELSSLRSEDTAMYYCNAAYGSSSYPMDYWGQGTTVTV

SEQ ID NO: 13 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDSGDTEYAQKFQDRVTITRDRSMSTAYMELSSLRSEDTAMYYCNAAYGSSSYPMDYWGQGTTVTV

SEQ ID NO: 14 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDNADTEYAQKFQDRVTITRDRSMSTAYMELSSLRSEDTAMYYCNAAYGSSSYPMDYWGQGTTVTV

SEQ ID NO: 15 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDNTDTEYAQKFQDRVTITRDRSMSTAYMELSSLRSEDTAMYYCNAAYGSSSYPMDYWGQGTTVTV

SEQ ID NO: 16 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDNGDTEYAQKFQDRVTITRDRSMSTAYMELSSLRSEDTAMYYCNAAYGSSPYPMDYWGQGTTVTV

SEQ ID NO: 17 - protein, 118 aa, synthetic construct
  REGION 1..118: note = Synthetic polypeptide
QMQLVQSGAEVKKTGSSVKVSCKASGYTFTYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 18 - protein, 118 aa, synthetic construct
  REGION 1..118: note = Synthetic polypeptide
QMQLVQSGAEVKKTGSSVKVSCKASGFTFTYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 19 - protein, 118 aa, synthetic construct
  REGION 1..118: note = Synthetic polypeptide
QMQLVQSGAEVKKTGSSVKVSCKASGYNFTYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 20 - protein, 118 aa, synthetic construct
  REGION 1..118: note = Synthetic polypeptide
QMQLVQSGAEVKKTGSSVKVSCKASGYTITYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 21 - protein, 118 aa, synthetic construct
  REGION 1..118: note = Synthetic polypeptide
QMQLVQSGAEVKKTGSSVKVSCKASGYTFKYYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 22 - protein, 118 aa, synthetic construct
  REGION 1..118: note = Synthetic polypeptide
QMQLVQSGAEVKKTGSSVKVSCKASGYTFTDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 23 - protein, 118 aa, synthetic construct
  REGION 1..118: note = Synthetic polypeptide
QMQLVQSGAEVKKTGSSVKVSCKASGFTFTDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 24 - protein, 118 aa, synthetic construct
  REGION 1..118: note = Synthetic polypeptide
QMQLVQSGAEVKKTGSSVKVSCKASGFTITDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 25 - protein, 118 aa, synthetic construct
  REGION 1..118: note = Synthetic polypeptide
QMQLVQSGAEVKKTGSSVKVSCKASGYTFKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 26 - protein, 118 aa, synthetic construct
  REGION 1..118: note = Synthetic polypeptide
QMQLVQSGAEVKKTGSSVKVSCKASGFTFKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 27 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDNGDTEYAQKFQDRVTITRDRSMSTAYLQLSSLRSEDTAMYYCNAAYGSSSYPMDYWGQGTTVTV

SEQ ID NO: 28 - protein, 118 aa, synthetic construct
  REGION 1..118: note = Synthetic polypeptide
QMQLVQSGAEVKKTGSSVKVSCKASGFNIKDYYLHWVRQAPGQALEWMGWIDPDNGDTEYAQKFQDRVTITRDRSMSTAYMELSSLTSEDTAVYYCNAAYGSSSYPMDYWGQGTTVTV

SEQ ID NO: 29 - protein, 118 aa, synthetic construct
  REGION 1..118: note = Synthetic polypeptide
EVQLVQSGAEVKKPGATVKISCKVSGFNIKDYYLHWVRQAPGQALEWMGWIDPDNGDTEYAQKFQDRVTITRDRSMSTAYMELSSLRSEDTAMYYCNAAYGSSSYPMDYWGQGTTVTV

SEQ ID NO: 30 - protein, 118 aa, synthetic construct
  REGION 1..118: note = Synthetic polypeptide
EVQLVQSGAEVKKPGATVKISCKVSGFNIKDYYLHWVQQAPGKGLEWMGWIDPDNGDTEYAQKFQDRVTITRDRSMSTAYMELSSLRSEDTAMYYCNAAYGSSSYPMDYWGQGTTVTV

SEQ ID NO: 31 - protein, 107 aa, synthetic construct
  REGION 1..107: note = Synthetic polypeptide
DIKMTQSPSSLYASLGERVTITCKASQDIHRYLSWFQQKPGKSPKILIYRANRLVDGVPSRFSGSGSGQDYSLTISSLEYEDMGIYYCLQYDEFPYTFGGGTKLEMK

SEQ ID NO: 32 - protein, 107 aa, synthetic construct
  REGION 1..107: note = Synthetic polypeptide
DIKMTQSPSSLYASLGERVTITCKASQDIHRYLSWFQQKPGKSPKILIYRANRLVDGVPSRFSGSGSGQDYSLTISSLEYEDMGIYYCLQYDEFPYTFGGGTKLEIK

SEQ ID NO: 33 - protein, 107 aa, synthetic construct
  REGION 1..107: note = Synthetic polypeptide
DIQMTQSPSSLSASVGDRVTITCKASQDIHRYLSWYQQKPGKAPKLLIYRANRLVDGVPSRFSGSGSGTDFTFTISSLQPEDIATYYCLQYDEFPYTFGGGTKVEIK

SEQ ID NO: 34 - protein, 107 aa, synthetic construct
  REGION 1..107: note = Synthetic polypeptide
DIQMTQSPSSLSASVGDRVTITCKASQDIHRYLSWFQQKPGKAPKSLIYRANRLVDGVPSRFSGSGSGTDFTLTISSLQPEDFATYYCLQYDEFPYTFGGGTKVEIK

SEQ ID NO: 35 - protein, 107 aa, synthetic construct
  REGION 1..107: note = Synthetic polypeptide
NIQMTQSPSAMSASVGDRVTITCKASQDIHRYLSWFQQKPGKVPKHLIYRANRLVDGVPSRFSGSGSGTEFTLTISSLQPEDFATYYCLQYDEFPYTFGGGTKVEIK

SEQ ID NO: 36 - protein, 107 aa, synthetic construct
  REGION 1..107: note = Synthetic polypeptide
DIQMTQSPSSLSASVGDRVTITCKASQDIHRYLSWYQQKPGKAPKRLIYRANRLVDGVPSRFSGSGSGTEFTLTISSLQPEDFATYYCLQYDEFPYTFGGGTKVEIK

SEQ ID NO: 37 - protein, 107 aa, synthetic construct
  REGION 1..107: note = Synthetic polypeptide
DIQMTQSPSSLSASVGDRVTITCRASQDIHRYLAWYQQKPGKVPKLLIYRANRLQSGVPSRFSGSGSGTDFTLTISSLQPEDVATYYCLQYDEFPYTFGQGTKVEIK

SEQ ID NO: 38 - protein, 107 aa, synthetic construct
  REGION 1..107: note = Synthetic polypeptide
EIVLTQSPATLSLSPGERATLSCRASQDIHRYLAWYQQKPGQAPRLLIYRANRRATGIPARFSGSGSGTDFTLTISSLEPEDFAVYYCLQYDEFPYTGFQGTRLEIK

SEQ ID NO: 39 - protein, 107 aa, synthetic construct
  REGION 1..107: note = Synthetic polypeptide
DIQMTQSPSAMSASVGDRVTITCKASQDIHRYLSWFQQKPGKVPKHLIYRANRLVDGVPSRFSGSGSGTEFTLTISSLQPEDFATYYCLQYDEFPYTFGGGTKVEIK

SEQ ID NO: 40 - protein, 107 aa, synthetic construct
  REGION 1..107: note = Synthetic polypeptide
NIQMTQSPSAMSASVGDRVTITCRARQGIHRYLSWFQQKPGKVPKHLIYRANRLVDGVPSRFSGSGSGTEFTLTISSLQPEDFATYYCLQYDEFPYTFGGGTKVEIK

SEQ ID NO: 41 - protein, 107 aa, synthetic construct
  REGION 1..107: note = Synthetic polypeptide
NIQMTQSPSAMSASVGDRVTITCKASQDIHRYLSWFQQKPGKVPKILIYRANRLVDGVPSRFSGSGSGTEFTLTISSLQPEDFATYYCLQYDEFPYTFGGGTKVEIK

SEQ ID NO: 42 - protein, 107 aa, synthetic construct
  REGION 1..107: note = Synthetic polypeptide
NIQMTQSPSAMSASVGDRVTITCKASQDIHRYLSWFQQKPGKVPKHLIYRANRLVSGVPSRFSGSGSGTEFTLTISSLQPEDFATYYCLQYDEFPYTFGGGTKVEIK

SEQ ID NO: 43 - protein, 107 aa, synthetic construct
  REGION 1..107: note = Synthetic polypeptide
NIQMTQSPSAMSASVGDRVTITCRARQGIHRYLSWFQQKPGKVPKILIYRANRLVDGVPSRFSGSGSGTEFTLTISSLQPEDFATYYCLQYDEFPYTFGGGTKVEIK

SEQ ID NO: 44 - protein, 107 aa, synthetic construct
  REGION 1..107: note = Synthetic polypeptide
NIQMTQSPSAMSASVGDRVTITCRARQGIHRYLSWFQQKPGKVPKHLIYRANRLVSGVPSRFSGSGSGTEFTLTISSLQPEDFATYYCLQYDEFPYTFGGGTKVEIK

SEQ ID NO: 45 - protein, 107 aa, synthetic construct
  REGION 1..107: note = Synthetic polypeptide
NIQMTQSPSAMSASVGDRVTITCKASQDIHRYLSWFQQKPGKVPKLLIYRANRLVDGVPSRFSGSGSGTEFTLTISSLQPEDFATYYCLQYDEFPYTFGGGTKVEIK

SEQ ID NO: 46 - protein, 107 aa, synthetic construct
  REGION 1..107: note = Synthetic polypeptide
NIQMTQSPSAMSASVGDRVTITCKASQDIHRYLSWFQQKPGKVPKLLIYRANRLVSGVPSRFSGSGSGTEFTLTISSLQPEDFATYYCLQYDEFPYTFGGGTKVEIK

SEQ ID NO: 47 - protein, 107 aa, synthetic construct
  REGION 1..107: note = Synthetic polypeptide
NIQMTQSPSAMSASVGDRVTITCRARQGIHRYLSWFQQKPGKVPKLLIYRANRLVSGVPSRFSGSGSGTEFTLTISSLQPEDFATYYCLQYDEFPYTFGGGTKVEIK

SEQ ID NO: 48 - protein, 323 aa, Homo sapiens
  REGION 1..323: note = human CD47 (Accession No. Q08722.1)
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 49 - protein, 305 aa, Homo sapiens
  REGION 1..305: note = human CD47
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 50 - protein, 10 aa, synthetic construct
  REGION 1..10: note = Synthetic polypeptide
GFNIKDYYLH

SEQ ID NO: 51 - protein, 10 aa, synthetic construct
  REGION 1..10: note = Synthetic polypeptide
WIDPDNGDTE

SEQ ID NO: 52 - protein, 13 aa, synthetic construct
  REGION 1..13: note = Synthetic polypeptide
NAAYGSSSYPMDY

SEQ ID NO: 53 - protein, 11 aa, synthetic construct
  REGION 1..11: note = Synthetic polypeptide
KASQDIHRYLS

SEQ ID NO: 54 - protein, 7 aa, synthetic construct
  REGION 1..7: note = Synthetic polypeptide
RANRLVD

SEQ ID NO: 55 - protein, 9 aa, synthetic construct
  REGION 1..9: note = Synthetic polypeptide
LQYDEFPYT

SEQ ID NO: 56 - protein, 4 aa, synthetic construct
  REGION 1..4: note = Synthetic polypeptide
KGRD

SEQ ID NO: 57 - protein, 10 aa, synthetic construct
  REGION 1..10: note = Synthetic polypeptide
GYTFTYYYLH

SEQ ID NO: 58 - protein, 10 aa, synthetic construct
  REGION 1..10: note = Synthetic polypeptide
GFTFTYYYLH

SEQ ID NO: 59 - protein, 10 aa, synthetic construct
  REGION 1..10: note = Synthetic polypeptide
GYNFTYYYLH

SEQ ID NO: 60 - protein, 10 aa, synthetic construct
  REGION 1..10: note = Synthetic polypeptide
GYTITYYYLH

SEQ ID NO: 61 - protein, 10 aa, synthetic construct
  REGION 1..10: note = Synthetic polypeptide
GYTFKYYYLH

SEQ ID NO: 62 - protein, 10 aa, synthetic construct
  REGION 1..10: note = Synthetic polypeptide
GYTFTDYYLH

SEQ ID NO: 63 - protein, 10 aa, synthetic construct
  REGION 1..10: note = Synthetic polypeptide
GFTFTDYYLH

SEQ ID NO: 64 - protein, 10 aa, synthetic construct
  REGION 1..10: note = Synthetic polypeptide
GFTITDYYLH

SEQ ID NO: 65 - protein, 10 aa, synthetic construct
  REGION 1..10: note = Synthetic polypeptide
GYTFKDYYLH

SEQ ID NO: 66 - protein, 10 aa, synthetic construct
  REGION 1..10: note = Synthetic polypeptide
GFTFKDYYLH

SEQ ID NO: 67 - protein, 11 aa, synthetic construct
  REGION 1..11: note = Synthetic polypeptide
RASQDIHRYLA

SEQ ID NO: 68 - protein, 11 aa, synthetic construct
  REGION 1..11: note = Synthetic polypeptide
RARQGIHRYLS

SEQ ID NO: 69 - protein, 7 aa, synthetic construct
  REGION 1..7: note = Synthetic polypeptide
RANRLQS

SEQ ID NO: 70 - protein, 7 aa, synthetic construct
  REGION 1..7: note = Synthetic polypeptide
RANRRAT

SEQ ID NO: 71 - protein, 7 aa, synthetic construct
  REGION 1..7: note = Synthetic polypeptide
RANRLVS

SEQ ID NO: 72 - protein, 10 aa, synthetic construct
  REGION 1..10: note = Synthetic polypeptide
WIDPDQGDTE

SEQ ID NO: 73 - protein, 10 aa, synthetic construct
  REGION 1..10: note = Synthetic polypeptide
WIDPDYGDTE

SEQ ID NO: 74 - protein, 10 aa, synthetic construct
  REGION 1..10: note = Synthetic polypeptide
WIDPDSGDTE

SEQ ID NO: 75 - protein, 10 aa, synthetic construct
  REGION 1..10: note = Synthetic polypeptide
WIDPDNADTE

SEQ ID NO: 76 - protein, 10 aa, synthetic construct
  REGION 1..10: note = Synthetic polypeptide
WIDPDNTDTE

SEQ ID NO: 77 - protein, 13 aa, synthetic construct
  REGION 1..13: note = Synthetic polypeptide
NAAYGSSPYPMDY

SEQ ID NO: 78 - protein, 451 aa, synthetic construct
  REGION 1..451: note = Synthetic polypeptide
MQMQLVQSGAEVKKTGSSVKVSCKASGFNIKDYYLHWVRQAPGQALEWMGWIDPDQGDTEYAQKFQDRVTITRDRSMSTAYMELSSLRSEDTAMYYCNAAYGSSSYPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 448 aa, synthetic construct
  REGION 1..448: note = Synthetic polypeptide
MQMQLVQSGAEVKKTGSSVKVSCKASGFNIKDYYLHWVRQAPGQALEWMGWIDPDQGDTEYAQKFQDRVTITRDRSMSTAYMELSSLRSEDTAMYYCNAAYGSSSYPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 448 aa, synthetic construct
  REGION 1..448: note = Synthetic polypeptide
MQMQLVQSGAEVKKTGSSVKVSCKASGFNIKDYYLHWVRQAPGQALEWMGWIDPDQGDTEYAQKFQDRVTITRDRSMSTAYMELSSLRSEDTAMYYCNAAYGSSSYPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 451 aa, synthetic construct
  REGION 1..451: note = Synthetic polypeptide
MQVQLVQSGAEVKKPGASVKVSCKASGFNIKDYYLHWVRQAPGQGLEWMGWIDPDQGDTEYAQKLQGRVTMTTDTSTSTAYMELRSLRSDDTAVYYCNAAYGSSSYPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 451 aa, synthetic construct
  REGION 1..451: note = Synthetic polypeptide
MQVQLVQSGAEVKKPGASVKVSCKASGFNIKDYYLHWVRQAPGQGLEWMGWIDPDQGDTEYAQKLQGRVTMTTDTSTSTAYMELRSLRSDDTAVYYCNAAYGSSSYPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 451 aa, synthetic construct
  REGION 1..451: note = Synthetic polypeptide
MQMQLVQSGAEVKKPGSSVKVSCKASGFNIKDYYLHWVRQAPGQALEWMGWIDPDQGDTEYAQKFQGRVTITRDRSTSTAYMELRSLRSEDTAVYYCNAAYGSSSYPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 448 aa, synthetic construct
  REGION 1..448: note = Synthetic polypeptide
MQVQLVQSGAEVKKPGASVKVSCKASGFNIKDYYLHWVRQAPGQGLEWMGWIDPDQGDTEYAQKLQGRVTMTTDTSTSTAYMELRSLRSDDTAVYYCNAAYGSSSYPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 448 aa, synthetic construct
  REGION 1..448: note = Synthetic polypeptide
MQMQLVQSGAEVKKPGSSVKVSCKASGFNIKDYYLHWVRQAPGQALEWMGWIDPDQGDTEYAQKFQGRVTITRDRSTSTAYMELRSLRSEDTAVYYCNAAYGSSSYPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 448 aa, synthetic construct
  REGION 1..448: note = Synthetic polypeptide
MQVQLVQSGAEVKKPGASVKVSCKASGFNIKDYYLHWVRQAPGQGLEWMGWIDPDQGDTEYAQKLQGRVTMTTDTSTSTAYMELRSLRSDDTAVYYCNAAYGSSSYPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 448 aa, synthetic construct
  REGION 1..448: note = Synthetic polypeptide
MQMQLVQSGAEVKKPGSSVKVSCKASGFNIKDYYLHWVRQAPGQALEWMGWIDPDQGDTEYAQKFQGRVTITRDRSTSTAYMELRSLRSEDTAVYYCNAAYGSSSYPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 215 aa, synthetic construct
  REGION 1..215: note = Synthetic polypeptide
MNIQMTQSPSAMSASVGDRVTITCKASQDIHRYLSWFQQKPGKVPKHLIYRANRLVSGVPSRFSGSGSGTEFTLTISSLQPEDFATYYCLQYDEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 119 aa, synthetic construct
  REGION 1..119: note = Synthetic polypeptide
  VARIANT 1: note = Xaa can be Met or none
  VARIANT 3: note = Xaa can be an amino acid with hydrophobic side chains such as Met or Val
  VARIANT 15: note = Xaa = Thr or Pro
  VARIANT 17: note = Xaa = Ser or Ala
  VARIANT 45: note = Xaa can be aliphatic side chains such as Ala or Gly
  VARIANT 65: note = Xaa = Phe or Leu
  VARIANT 67: note = Xaa = Asp or Gly
  VARIANT 71: note = Xaa can be an amino acid with hydrophobic side chains such as Ile or Met
  VARIANT 73: note = Xaa = Arg or Thr
  VARIANT 75: note = Xaa = Arg or Thr
  VARIANT 77: note = Xaa = Met or Thr
  VARIANT 85: note = Xaa = Ser or Arg
  VARIANT 90: note = Xaa can be a negatively charged amino acid such as Glu or Asp
  VARIANT 94: note = Xaa can be an amino acid with hydrophobic side chains such as Met or Val
XQXQLVQSGAEVKKXGXSVKVSCKASGFNIKDYYLHWVRQAPGQXLEWMGWIDPDQGDTEYAQKXQXRVTXTXDXSXSTAYMELXSLRSXDTAXYYCNAAYGSSSYPMDYWGQGTTVTV

SEQ ID NO: 90 - protein, 119 aa, synthetic construct
  REGION 1..119: note = Synthetic polypeptide
MQVQLVQSGAEVKKPGASVKVSCKASGFNIKDYYLHWVRQAPGQGLEWMGWIDPDQGDTEYAQKLQGRVTMTTDTSTSTAYMELRSLRSDDTAVYYCNAAYGSSSYPMDYWGQGTTVTV

SEQ ID NO: 91 - protein, 119 aa, synthetic construct
  REGION 1..119: note = Synthetic polypeptide
MQMQLVQSGAEVKKPGSSVKVSCKASGFNIKDYYLHWVRQAPGQALEWMGWIDPDQGDTEYAQKLQGRVTMTTDTSTSTAYMELRSLRSDDTAVYYCNAAYGSSSYPMDYWGQGTTVTV

(19) United States
(12) Patent Application Publication
(10) Pub. No.: US 2024/0075133 A1
(43) Pub. Date: Mar. 7, 2024

(54) METHODS AND COMPOSITIONS FOR REDUCTION OF IMMUNOGENICITY

(71) Applicant: CELGENE CORPORATION, Summit, NJ (US)

(72) Inventors: Jeffrey C. Johnson, Carlsbad, CA (US); Lawrence Dearth, Encinitas, CA (US); Haralambos HADJIVASSILIOU, San Diego, CA (US); Jeonghoon Sun, San Diego, CA (US); Kandasamy Hariharan, San Diego, CA (US)

(21) Appl. No.: 18/335,656

(22) Filed: Jun. 15, 2023

Related U.S. Application Data
(62) Division of application No. 16/498,063, filed on Sep. 26, 2019, now abandoned.
(60) Provisional application No. 62/477,257, filed on Mar. 27, 2017.

(51) Int. Cl. A61K 39/395; A61K 38/20; A61K 45/06

(52) U.S. Cl. CPC A61K 39/3955; A61K 38/20; A61K 45/06

(57) ABSTRACT

Provided herein are methods and uses involving the combination of an anti-CD20 antibody, e.g., rituximab with a protein therapeutic, for example, an antibody (e.g., an antibody that specifically binds to human CD47).

### CROSS-REFERENCE TO RELATED APPLICATIONS

This application is a Divisional of U.S. application Ser. No. 16/498,063, filed Sep. 26, 2019, which is a U.S. National Phase of International Application No. PCT/US2018/024316, filed Mar. 26, 2018, which claims priority to U.S. Provisional Patent Application No. 62/477,257, filed Mar. 27, 2017, each of which are incorporated herein by reference in their entirety.

### SEQUENCE LISTING

The contents of the electronic sequence listing (298068-00423.xml; Size: 131,142 bytes; and Date of Creation: Nov. 6, 2023) is herein incorporated by reference in its entirety.

### 1. FIELD

Provided herein are methods and uses involving the combination of an anti-CD20 antibody, e.g., rituximab with a protein therapeutic, for example, an antibody (e.g., an antibody that specifically binds to human CD47).

### 2. BACKGROUND

While protein therapeutics have been used to treat a number of diseases, they can prompt an immune response involving production of anti-drug antibodies when administered to subjects. This can result in reduced efficacy of the therapeutic and/or toxicity. Accordingly, there exists a need for methods of reducing this immune response.

CD47, also known as integrin-associated protein (IAP), ovarian cancer antigen OA3, Rh-related antigen and MERG, is a multi-spanning transmembrane receptor belonging to the immunoglobulin superfamily SIRPα (signal-regulatory-protein a) expressed on macrophages interacts with CD47, and this interaction negatively controls effector function of innate immune cells such as host cell phagocytosis. CD47 expression and/or activity have been implicated in a number of diseases and disorders. Accordingly, there exists a need for therapies that target CD47.

### 3. SUMMARY

Provided herein are methods and compositions for reducing immunogenicity in a subject, comprising administering to a subject an anti-CD20 antibody, e.g., rituximab, in combination with a protein therapeutic, wherein the immunogenicity is reduced in comparison with the immunogenicity in a subject when the protein therapeutic is administered alone. In certain aspects, the protein therapeutic is an antibody therapeutic. In certain aspects, the protein therapeutic is a fusion protein, for example, an Fc-containing fusion protein, e.g., a soluble receptor fusion protein. In certain aspects, the protein therapeutic is a cytokine. In certain aspects, the protein therapeutic is an interleukin. In certain aspects, the protein therapeutic is not an enzyme. In certain aspects, the subject is a human

In certain aspects of the methods and compositions provided herein, the protein therapeutic is an antibody, wherein the antibody therapeutic is an antibody that binds to CD47 or an antigen-binding fragment thereof.

In specific aspects, the antibody that binds to CD47 or antigen-binding fragment thereof comprises a VH CDR1 comprising SEQ ID NO: 50, a VH CDR2 comprising SEQ ID NO: 72, a VH CDR3 comprising SEQ ID NO: 52, a VL CDR1 comprising SEQ ID NO: 53, a VL CDR2 comprising SEQ ID NO: 71, and a VL CDR3 comprising SEQ ID NO: 55.

In specific aspects, the antibody that binds to CD47 or antigen-binding fragment thereof comprises a VH CDR1 comprising SEQ ID NO: 50, a VH CDR2 comprising SEQ ID NO: 51, a VH CDR3 comprising SEQ ID NO: 52, a VL CDR1 comprising SEQ ID NO: 53, a VL CDR2 comprising SEQ ID NO: 54, and a VL CDR3 comprising SEQ ID NO: 55.

In certain aspects, the antibody that binds to CD47 or antigen-binding fragment thereof comprises a VH comprising a sequence selected from the group consisting of SEQ ID NOs: 5-30. In certain aspects, the antibody that binds to CD47 or antigen-binding fragment thereof comprises a VL comprising a sequence selected from the group consisting of SEQ ID NOs: 31-47. In certain aspects, the antibody that binds to CD47 or antigen-binding fragment thereof comprises a VH comprising a sequence selected from the group consisting of SEQ ID NOs: 5-30 and a VL comprising a sequence selected from the group consisting of SEQ ID NOs: 31-47.

In certain aspects, the antibody that binds to CD47 or antigen-binding fragment thereof is an IgG isotype selected from the group consisting of IgG1 isotype, IgG2 isotype, IgG3 isotype, and IgG4 isotype. In certain aspects, the antibody that binds to CD47 or antigen-binding fragment thereof is an IgG isotype selected from IgG4P and IgG4PE.

In certain aspects, the antibody that binds to CD47 or antigen-binding fragment thereof is a component of a pharmaceutical composition comprising the antibody that binds to CD47 or an antigen-binding fragment thereof and a pharmaceutically acceptable carrier.

In certain aspects, the antibody that binds to CD47 or antigen-binding fragment thereof is a chimeric, humanized, or fully human antibody.

In certain aspects, the methods provided herein additionally comprise administering a second therapeutic, e.g., a small molecule therapeutic, such as a chemotherapy therapeutic. In specific aspects, said chemotherapy is radiotherapy.

In certain aspects, the antibody that binds to CD47 or antigen-binding fragment thereof is administered to the subject at a dose of 0.3, 1, 2, 4, 8, 15, or 20 mg/kg. In certain aspects, the anti-CD20 antibody, e.g., rituximab, is administered to the subject at a dose of 300, 325, 350, 375, 400, 425, 450, or 500 mg/m². In certain aspects, the anti-CD20 antibody is rituximab In certain aspects, the antibody that binds to CD47 or antigen-binding fragment thereof is administered to the subject at a dose of 0.3, 1, 2, 4, 8, 15, or 20 mg/kg and the anti-CD20 antibody, e.g., rituximab, is administered to the subject at a dose of 300, 325, 350, 375, 400, 425, 450, or 500 mg/m². In certain aspects, the anti-CD20 antibody, e.g., rituximab, is administered prior to the protein therapeutic. In certain aspects, the anti-CD20 antibody, e.g., rituximab, is administered 1, 2, 3, 4, 5, or 6 weeks prior to the protein therapeutic. In certain aspects, the anti-CD20 antibody, e.g., rituximab, is administered 1, 2, 3, 4, 5, or 6 days prior to the protein therapeutic. In certain aspects, the rituximab is administered prior to the antibody that binds to CD47 or antigen-binding fragment thereof. In certain aspects, the anti-CD20 antibody, e.g., rituximab, is administered 1, 2, 3, 4, 5, or 6 weeks prior to the antibody that binds to CD47 or antigen-binding fragment thereof. In certain aspects, the anti-CD20 antibody, e.g., rituximab, is administered 1, 2, 3, 4, 5, or 6 days prior to the antibody that binds to CD47 or antigen-binding fragment thereof.

In certain aspects, the methods provided herein do not comprise administering a proteosome inhibitor to the subject. In certain aspects, the methods provided herein do not comprise administering bortezomib to the subject. In certain aspects, the methods provided herein do not comprise administering methotrexate to the subject.

Provided herein are methods for treating cancer, the method comprising administering to a subject in need thereof a therapeutically effective amount of an antibody that binds to CD47 or an antigen-binding fragment thereof, wherein the method additionally comprises administering an anti-CD20 antibody, e.g., rituximab, to the subject. In certain aspects, the anti-CD20 antibody is rituximab. In certain aspects, the subject is a human. In certain aspects, the methods provided herein further comprise administering radiation or chemotherapy. In certain aspects, the methods provided herein further comprise administering another anti-cancer agent. In certain aspects, the cancer is a hematological cancer. In certain aspects, the cancer is a solid cancer. In certain aspects, the cancer is multiple myeloma, non-Hodgkin's lymphoma, acute myeloid leukemia (AML), breast cancer (e.g., triple negative breast cancer), bladder cancer, non-small cell lung cancer/carcinoma, hepatocellular carcinoma (HCC), sarcoma, or head and neck cancer. In certain aspects, the cancer is multiple myeloma. In certain aspects, the cancer is non-Hodgkin's lymphoma. In specific aspects, the non-Hodgkin's lymphoma is CD20 positive. In specific aspects, the non-Hodgkin's lymphoma is relapsed or refractory. In specific aspects, the subject has previously received a therapeutic regimen including anti-CD20 antibody, e.g., rituximab.

Provided herein are methods for treating cancer, the method comprising administering to a subject in need thereof a therapeutically effective amount of an antibody that binds to CD47 or an antigen-binding fragment thereof. In certain aspects, the subject is a human In certain aspects, the methods provided herein further comprise administering radiation or chemotherapy. In certain aspects, the methods provided herein further comprise administering another anti-cancer agent. In certain aspects, the cancer is a hematological cancer. In certain aspects, the cancer is a solid cancer. In certain aspects, the cancer is multiple myeloma, non-Hodgkin's lymphoma, acute myeloid leukemia (AML), breast cancer (e.g., triple negative breast cancer), bladder cancer, non-small cell lung cancer/carcinoma, hepatocellular carcinoma (HCC), sarcoma, or head and neck cancer. In certain aspects, the cancer is multiple myeloma. In certain aspects, the cancer is non-Hodgkin's lymphoma. In specific aspects, the non-Hodgkin's lymphoma is CD20 positive. In specific aspects, the non-Hodgkin's lymphoma is relapsed or refractory. In specific aspects, the subject has previously received a therapeutic regimen including anti-CD20 antibody, e.g., rituximab.

In certain aspects, the antibody that binds to CD47 or antigen-binding fragment thereof is administered to the subject at a dose of 0.3, 1, 2, 4, 8, 15, or 20 mg/kg. In certain aspects, the anti-CD20 antibody, e.g., rituximab, is administered to the subject at a dose of 300, 325, 350, 375, 400, 425, 450, or 500 mg/m². In certain aspects, the anti-CD20 antibody, e.g., rituximab, is administered prior to the antibody that binds to CD47 or antigen-binding fragment thereof.

In certain aspects, the method does not comprise administering a proteosome inhibitor to the subject. In certain aspects, the method does not comprise administering bortezomib to the subject. In certain aspects, the method does not comprise administering methotrexate to the subject.

In certain aspects of the methods of treating cancer provided herein, the protein therapeutic is an antibody, wherein the antibody therapeutic is an antibody that binds to CD47 or an antigen-binding fragment thereof.

In specific aspects, the antibody that binds to CD47 or antigen-binding fragment thereof comprises a VH CDR1 comprising SEQ ID NO: 50, a VH CDR2 comprising SEQ ID NO: 72, a VH CDR3 comprising SEQ ID NO: 52, a VL CDR1 comprising SEQ ID NO: 53, a VL CDR2 comprising SEQ ID NO: 71, and a VL CDR3 comprising SEQ ID NO: 55.

In specific aspects, the antibody that binds to CD47 or antigen-binding fragment thereof comprises a VH CDR1 comprising SEQ ID NO: 50, a VH CDR2 comprising SEQ ID NO: 51, a VH CDR3 comprising SEQ ID NO: 52, a VL CDR1 comprising SEQ ID NO: 53, a VL CDR2 comprising SEQ ID NO: 54, and a VL CDR3 comprising SEQ ID NO: 55.

In certain aspects, the antibody that binds to CD47 or antigen-binding fragment thereof comprises a VH comprising a sequence selected from the group consisting of SEQ ID NOs: 5-30. In certain aspects, the antibody that binds to CD47 or antigen-binding fragment thereof comprises a VL comprising a sequence selected from the group consisting of SEQ ID NOs: 31-47. In certain aspects, the antibody that binds to CD47 or antigen-binding fragment thereof comprises a VH comprising a sequence selected from the group consisting of SEQ ID NOs: 5-30 and a VL comprising a sequence selected from the group consisting of SEQ ID NOs: 31-47.

In certain aspects, the antibody that binds to CD47 or antigen-binding fragment thereof is an IgG isotype selected from the group consisting of IgG1 isotype, IgG2 isotype, IgG3 isotype, and IgG4 isotype. In certain aspects, the antibody that binds to CD47 or antigen-binding fragment thereof is an IgG isotype selected from IgG4P and IgG4PE. In certain aspects, the antibody that binds to CD47 or antigen-binding fragment thereof is chimeric, humanized, or fully human

### 4. DETAILED DESCRIPTION

### 4.1 Methods

### 4.1.1 Methods of Reducing Immunogenicity

Provided herein are methods for reducing immunogenicity in a subject, comprising administering to a subject an anti-CD20 antibody, e.g., rituximab, in combination with a protein therapeutic, wherein the immunogenicity is reduced in comparison with the immunogenicity in the subject when administering the protein therapeutic alone. In certain embodiments, the protein therapeutic is an antibody therapeutic. In certain embodiments, the protein therapeutic is a cytokine. In certain embodiments, the cytokine is bone morphogenetic protein (BMP), erythropoietin (EPO), granulocyte colony-stimulating factor (G-CSF), granulocyte macrophage colony-stimulating factor (GM-CSF), interferon alpha (IFN-α), interferon beta (IFN-β), interleukin 2 (IL-2), interleukin 11 (IL-11), or interferon gamma (IFN-γ). In certain embodiments, the protein therapeutic is an interleukin. In certain embodiments, the protein therapeutic is not an enzyme.

In certain embodiments of the methods provided herein, the protein therapeutic is an antibody therapeutic, wherein the antibody therapeutic is an antibody that binds to CD47 or an antigen-binding fragment thereof.

In certain embodiments, the antibody that binds to CD47 or antigen-binding fragment thereof is a component of a pharmaceutical composition comprising the antibody that binds to CD47 or an antigen-binding fragment thereof and a pharmaceutically acceptable carrier.

In certain embodiments, the methods provided herein comprise administering chemotherapy. In specific embodiments, said chemotherapy is radiotherapy.

In certain embodiments, the anti-CD20 antibody, e.g., rituximab, is administered prior to and/or concurrently with the protein therapeutic. In certain embodiments, the anti-CD20 antibody, e.g., rituximab,b is administered prior to the protein therapeutic. In certain embodiments, the anti-CD20 antibody, e.g., rituximab, is administered concurrently with the protein therapeutic.

In certain embodiments, the anti-CD20 antibody, e.g., rituximab, is administered prior to and/or concurrently with the antibody that binds to CD47 or antigen-binding fragment thereof. In certain embodiments, the anti-CD20 antibody, e.g., rituximab, is administered prior to the antibody that binds to CD47 or antigen-binding fragment thereof. In certain embodiments, the anti-CD20 antibody, e.g., rituximab, is administered concurrently with the antibody that binds to CD47 or antigen-binding fragment thereof.

In certain embodiments, the methods provided herein do not comprise administering a proteosome inhibitor to the subject. In certain embodiments, the methods provided herein do not comprise administering bortezomib to the subject. In certain embodiments, the methods provided herein do not comprise administering methotrexate to the subject.

Immunogenicity may be measured by any method known to one of skill in the art. In certain embodiments, immunogenicity is measured by determining the number and/or concentration of anti-drug antibodies present in the serum. In certain embodiments, immunogenicity is measured by determining the titer of anti-drug antibodies present in the serum. In certain embodiments, immunogenicity is measured by determining the amount of protein therapeutic neutralized per volume of serum. In certain embodiments, the presence of immunogenicity is indicated by the occurrence of anaphylaxis, cytokine release syndrome, infusion reactions, delayed hypersensitivity, and/or cross-reactivity to endogenous proteins. In certain embodiments, immunogenicity is measured by a screening assay. In specific embodiments, the screening assay is a direct binding enzyme-linked immunosorbent assay (ELISA), a bridging ELISA, a radioimmunoprecipitation assay (RIPA), a surface plasmon resonance (SPR) assay, a Bethesda Assay, or a bridging electrochemiluminescence assay. In certain embodiments, immunogenicity is measured by a neutralization assay. In specific embodiments, the neutralization assay is a cell-based biologic assay or a non cell-based competitive ligand-binding assay. In certain embodiments, the anti-drug antibodies bind to the protein therapeutic. In certain embodiments, the anti-drug antibodies neutralize the protein therapeutic. In certain embodiments, the anti-drug antibodies bind to and neutralize the protein therapeutic.

In certain aspects, immunogenicity is measured one week after the first dose of the protein therapeutic. In certain aspects, immunogenicity is measured two weeks after the first dose of the protein therapeutic. In certain aspects, immunogenicity is measured three weeks after the first dose of the protein therapeutic. In certain aspects, immunogenicity is measured four weeks after the first dose of the protein therapeutic. In certain aspects, immunogenicity is measured five weeks after the first dose of the protein therapeutic. In certain aspects, immunogenicity is measured six weeks after the first dose of the protein therapeutic.

In certain aspects, immunogenicity is measured weekly after the first dose of the protein therapeutic. In certain aspects, immunogenicity is measured one week after the first dose of the protein therapeutic and weekly thereafter. In certain aspects, immunogenicity is measured two weeks after the first dose of the protein therapeutic and weekly thereafter. In certain aspects, immunogenicity is measured three weeks after the first dose of the protein therapeutic and weekly thereafter. In certain aspects, immunogenicity is measured four weeks after the first dose of the protein therapeutic and weekly thereafter. In certain aspects, immunogenicity is measured five weeks after the first dose of the protein therapeutic and weekly thereafter. In certain aspects, immunogenicity is measured six weeks after the first dose of the protein therapeutic and weekly thereafter. In certain aspects, immunogenicity is measured every other week after the first dose of the protein therapeutic.

In certain aspects, B cell count is measured one week after the first dose of the protein therapeutic. In certain aspects, B cell count is measured two weeks after the first dose of the protein therapeutic. In certain aspects, B cell count is measured three weeks after the first dose of the protein therapeutic. In certain aspects, B cell count is measured four weeks after the first dose of the protein therapeutic. In certain aspects, B cell count is measured five weeks after the first dose of the protein therapeutic. In certain aspects, B cell count is measured six weeks after the first dose of the protein therapeutic.

In certain aspects, B cell count is measured one week after the first dose of the protein therapeutic. In certain aspects, B cell count is measured two weeks after the first dose of the protein therapeutic. In certain aspects, B cell count is measured three weeks after the first dose of the protein therapeutic. In certain aspects, B cell count is measured four weeks after the first dose of the protein therapeutic. In certain aspects, B cell count is measured five weeks after the first dose of the protein therapeutic. In certain aspects, B cell count is measured six weeks after the first dose of the protein therapeutic.

In certain aspects, B cell count is measured weekly after the first dose of the protein therapeutic. In certain aspects, B cell count is measured one week after the first dose of the protein therapeutic and weekly thereafter. In certain aspects, B cell count is measured two weeks after the first dose of the protein therapeutic and weekly thereafter. In certain aspects, B cell count is measured three weeks after the first dose of the protein therapeutic and weekly thereafter. In certain aspects, B cell count is measured four weeks after the first dose of the protein therapeutic and weekly thereafter. In certain aspects, B cell count is measured five weeks after the first dose of the protein therapeutic and weekly thereafter. In certain aspects, B cell count is measured six weeks after the first dose of the protein therapeutic and weekly thereafter. In certain aspects, B cell count is measured every other week after the first dose of the protein therapeutic.

In certain aspects, immunogenicity is measured one week after the first dose of the protein therapeutic. In certain aspects, immunogenicity is measured two weeks after the first dose of the protein therapeutic. In certain aspects, immunogenicity is measured three weeks after the first dose of the protein therapeutic. In certain aspects, immunogenicity is measured four weeks after the first dose of the protein therapeutic. In certain aspects, immunogenicity is measured five weeks after the first dose of the protein therapeutic. In certain aspects, immunogenicity is measured six weeks after the first dose of the protein therapeutic.

In certain aspects, immunogenicity is measured weekly after the first dose of anti-CD20 antibody, e.g., rituximab In certain aspects, the anti-CD20 antibody is rituximab. In certain aspects, immunogenicity is measured one week after the first dose of rituximab and weekly thereafter. In certain aspects, immunogenicity is measured two weeks after the first dose of rituximab and weekly thereafter. In certain aspects, immunogenicity is measured three weeks after the first dose of rituximab and weekly thereafter. In certain aspects, immunogenicity is measured four weeks after the first dose of rituximab and weekly thereafter. In certain aspects, immunogenicity is measured five weeks after the first dose of rituximab and weekly thereafter. In certain aspects, immunogenicity is measured six weeks after the first dose of rituximab and weekly thereafter. In certain aspects, immunogenicity is measured every other week after the first dose of rituximab.

In certain aspects, B cell count is measured one week after the first dose of anti-CD20 antibody, e.g., rituximab In certain aspects, the anti-CD20 antibody is rituximab. In certain aspects, B cell count is measured two weeks after the first dose of rituximab In certain aspects, B cell count is measured three weeks after the first dose of rituximab. In certain aspects, B cell count is measured four weeks after the first dose of rituximab. In certain aspects, B cell count is measured five weeks after the first dose of rituximab In certain aspects, B cell count is measured six weeks after the first dose of rituximab.

In certain aspects, B cell count is measured one week after the first dose of anti-CD20 antibody, e.g., rituximab In certain aspects, the anti-CD20 antibody is rituximab. In certain aspects, B cell count is measured two weeks after the first dose of rituximab In certain aspects, B cell count is measured three weeks after the first dose of rituximab. In certain aspects, B cell count is measured four weeks after the first dose of rituximab. In certain aspects, B cell count is measured five weeks after the first dose of rituximab In certain aspects, B cell count is measured six weeks after the first dose of rituximab.

In certain aspects, B cell count is measured weekly after the first dose of anti-CD20 antibody, e.g., rituximab In certain aspects, the anti-CD20 antibody is rituximab. In certain aspects, B cell count is measured one week after the first dose of rituximab and weekly thereafter. In certain aspects, B cell count is measured two weeks after the first dose of rituximab and weekly thereafter. In certain aspects, B cell count is measured three weeks after the first dose of rituximab and weekly thereafter. In certain aspects, B cell count is measured four weeks after the first dose of rituximab and weekly thereafter. In certain aspects, B cell count is measured five weeks after the first dose of rituximab and weekly thereafter. In certain aspects, B cell count is measured six weeks after the first dose of rituximab and weekly thereafter. In certain aspects, B cell count is measured every other week after the first dose of rituximab

### 4.1.2 Methods of Treating Cancer

Provided herein are methods for treating cancer, the method comprising administering to a subject in need thereof a therapeutically effective amount of an antibody that binds to CD47 or an antigen-binding fragment thereof, wherein the method additionally comprises administering anti-CD20 antibody, e.g., rituximab to the subject. In certain embodiments, the anti-CD20 antibody is rituximab. Also provided herein are methods for treating cancer, the method comprising administering to a subject in need thereof a therapeutically effective amount of an antibody that binds to CD47 or an antigen-binding fragment thereof.

In certain embodiments, the methods for treating cancer provided herein further comprise administering radiation or chemotherapy. In certain embodiments, the methods provided herein further comprise administering another anti-cancer agent. In certain embodiments, the cancer is a hematological cancer. In certain embodiments, the cancer is a solid cancer. In certain embodiments, the cancer is multiple myeloma, non-Hodgkin's lymphoma, acute myeloid leukemia (AML), breast cancer (e.g., triple negative breast cancer), bladder cancer, non-small cell lung cancer/carcinoma, hepatocellular carcinoma (HCC), sarcoma, or head and neck cancer. In specific embodiments, the cancer is non-Hodgkin's lymphoma. In specific embodiments, the non-Hodgkin's lymphoma is CD20 positive. In specific embodiments, the non-Hodgkin's lymphoma is relapsed or refractory. In specific embodiments, the subject has previously been treated with an anti-CD20 antibody, e.g., rituximab.

In certain embodiments, the anti-CD20 antibody, e.g., rituximab, is administered prior to and/or concurrently with the antibody that binds to CD47 or antigen-binding fragment thereof. In certain embodiments, the anti-CD20 antibody is rituximab. In certain embodiments, the anti-CD20 antibody, e.g., rituximab, is administered prior to the antibody that binds to CD47 or antigen-binding fragment thereof. In certain embodiments, the anti-CD20 antibody, e.g., rituximab, is administered concurrently with the antibody that binds to CD47 or antigen-binding fragment thereof.

In certain embodiments, the method does not comprise administering a proteosome inhibitor to the subject. In certain embodiments, the method does not comprise administering bortezomib to the subject. In certain embodiments, the method does not comprise administering methotrexate to the subject. In certain embodiments, the method additionally comprises administering methotrexate to the subject.

In certain embodiments, the antibody that binds to CD47 or antigen-binding fragment thereof is a component of a pharmaceutical composition comprising the antibody that binds to CD47 or an antigen-binding fragment thereof and a pharmaceutically acceptable carrier.

In certain embodiments, provided herein are methods of protecting against a condition or disorder, such as cancer, using an anti-CD47 antibody described herein alone or in combination with an anti-CD20 antibody, e.g., rituximab

In particular embodiments, provided herein are methods for managing, treating, preventing or protecting against cancer, comprising administering to a subject in need thereof a therapeutically effective amount of an antibody or an antigen-binding fragment described herein that binds specifically to CD47 (e.g., human CD47) alone or in combination with an anti-CD20 antibody, e.g., rituximab. In certain embodiments, provided herein is a method of alleviating, inhibiting or reducing the progression or severity of one or more symptoms associated with cancer.

As used herein, “administer” or “administration” refers to the act of injecting or otherwise physically delivering a substance (e.g., anti-CD20 antibody, e.g., rituximab, or an anti-CD47 antibody provided herein, or an antigen-binding fragment thereof) to a subject or a patient (e.g., human), such as by mucosal, topical, intradermal, parenteral, intravenous, subcutaneous, intramuscular delivery and/or any other method of physical delivery described herein or known in the art. In certain embodiments, administration of the anti-CD20 antibody, e.g., rituximab, is performed intravenously. In certain embodiments, administration of an anti-CD47 antibody provided herein is performed intravenously. In certain embodiments, administration of rituximab and an anti-CD47 antibody provided herein is performed intravenously.

As used herein, the term “effective amount” refers to an amount of a composition (e.g., an antibody or pharmaceutical composition provided herein or an anti-CD20 antibody, e.g., rituximab, or a pharmaceutical composition provided herein) which is sufficient to achieve a specific readout, for example, reduce and/or ameliorate the severity and/or duration of a given condition, disorder or disease (e.g., cancer, metastasis, or angiogenesis) and/or a symptom related thereto. Such a term also encompasses an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy (e.g., a therapy other than an anti-CD47 antibody provided herein). In some embodiments, “effective amount” as used herein refers to the amount of the anti-CD20 antibody, e.g., rituximab, associated with a reduction in the immunogenicity of a protein therapeutic, as described herein.

Doses of anti-CD20 antibody, e.g., rituximab, for example, to be administered in combination with a protein therapeutic, such as an anti-CD47 antibody, may include about 0.1 mg/kg body weight to about 100 mg/kg body weight. Doses of rituximab, for example, to be administered in combination with a protein therapeutic, such as an anti-CD47 antibody, may include about 25 mg/m² to about 1500 mg/m². In some embodiments, rituximab is administered to a subject at a dose of 0.1 mg/kg, 0.5 mg/kg, 1 mg/kg, 2 mg/kg, 5 mg/kg, 10 mg/kg, 15 mg/kg, 20 mg/kg, 25 mg/kg, 30 mg/kg, 50 mg/kg, 75 mg/kg, 100 mg/kg, or greater. In certain embodiments, rituximab is administered to the subject at a dose of 0.1, 0.3, 0.5, 1, 2, 4, 5, 8, 10, 15, 20, 25, 30, 50, 75 or 100 mg/kg. In certain embodiments, rituximab is administered to the subject at a dose of 0.3, 1, 2, 4, 8, 15, or 20 mg/kg. In some embodiments, rituximab is administered to a subject at a dose of 25, 50, 75, 100, 125, 150, 175, 200, 225, 250, 275, 300, 325, 350, 375, 400, 425, 450, 475, 500, 750, 1000, 1250, 1500 mg/m², or greater. In certain embodiments, rituximab is administered to the subject at a dose of 300, 325, 350, 375, 400, 425, 450, 475, or 500 mg/m². In certain embodiments, rituximab is administered to the subject at a dose of 375 mg/m².

Dosing frequencies of anti-CD20 antibody, e.g., rituximab, may range, for example, from twice daily to once a month. In certain embodiments, rituximab is administered to the subject at a dose of 300, 325, 350, 375, 400, 425, 450, 475, or 500 mg/m² once weekly. In certain embodiments, the rituximab is administered to the subject at a dose of 300, 325, 350, 375, 400, 425, 450, 475, or 500 mg/m² every two weeks. In certain embodiments, the rituximab is administered to the subject at a dose of 300, 325, 350, 375, 400, 425, 450, 475, or 500 mg/m² every four weeks. In certain embodiments, rituximab is administered to the subject at a dose of 375 mg/m² once weekly. In certain embodiments, rituximab is administered to the subject at a dose of 375 mg/m² every two weeks. In certain embodiments, rituximab is administered to the subject at a dose of 375 mg/m² every four weeks.

Doses of the anti-CD47 antibody or antigen-binding fragment thereof, for example, to be administered in combination with anti-CD20 antibody, e.g., rituximab, may include about 0.1 mg/kg body weight to about 100 mg/kg body weight. In some embodiments, an anti-CD47 antibody is administered to a subject at a dose of 0.1 mg/kg, 0.5 mg/kg, 1 mg/kg, 2 mg/kg, 5 mg/kg, 10 mg/kg, 15 mg/kg, 20 mg/kg, 25 mg/kg, 30 mg/kg, 50 mg/kg, 75 mg/kg, 100 mg/kg, or greater. In certain embodiments, the antibody that binds to CD47 or antigen-binding fragment thereof is administered to the subject at a dose of 0.1, 0.3, 0.5, 1, 2, 4, 5, 8, 10, 15, 20, 25, 30, 50, 75 or 100 mg/kg. In certain embodiments, the antibody that binds to CD47 or antigen-binding fragment thereof is administered to the subject at a dose of 0.3, 1, 2, 4, 8, 15, or 20 mg/kg.

Dosing frequencies of the anti-CD47 antibody or antigen-binding fragment thereof may range, for example, from twice daily to once a week. In certain embodiments, the antibody that binds to CD47 or antigen-binding fragment thereof is administered to the subject at a dose of 0.3, 1, 2, 4, 8, 15, or 20 mg/kg once weekly. In certain embodiments, the antibody that binds to CD47 or antigen-binding fragment thereof is administered to the subject at a dose of 0.3, 1, 2, 4, 8, 15, or 20 mg/kg twice weekly. In certain embodiments, the antibody that binds to CD47 or antigen-binding fragment thereof is administered to the subject at a dose of 8 mg/kg twice weekly. In certain embodiments, the antibody that binds to CD47 or antigen-binding fragment thereof is administered to the subject at a dose of 20 mg/kg once weekly.

In some embodiments, anti-CD20 antibody, e.g., rituximab, is administered to a subject at a dose of 25, 50, 75, 100, 125, 150, 175, 200, 225, 250, 275, 300, 325, 350, 375, 400, 425, 450, 475, 500, 750, 1000, 1250, 1500 mg/m², and the antibody that binds to CD47 or antigen-binding fragment thereof is administered to the subject at a dose of 0.1, 0.3, 0.5, 1, 2, 4, 5, 8, 10, 15, 20, 25, 30, 50, 75 or 100 mg/kg. In certain embodiments, rituximab is administered to the subject at a dose of 300, 325, 350, 375, 400, 425, 450, 475, or 500 mg/m² and the antibody that binds to CD47 or antigen-binding fragment thereof is administered to the subject at a dose of 0.3, 1, 2, 4, 8, 15, or 20 mg/kg. In certain embodiments, rituximab is administered to the subject at a dose of 375 mg/m² and the antibody that binds to CD47 or antigen-binding fragment thereof is administered to the subject at a dose of 0.3, 1, 2, 4, 8, 15, or 20 mg/kg. In certain embodiments, rituximab is administered to the subject at a dose of 300, 325, 350, 375, 400, 425, 450, 475, or 500 mg/m² once weekly and the antibody that binds to CD47 or antigen-binding fragment thereof is administered to the subject at a dose of 0.3, 1, 2, 4, 8, 15, or 20 mg/kg once weekly.

In certain embodiments, the anti-CD20 antibody, e.g., rituximab, is administered to the subject at a dose of 300, 325, 350, 375, 400, 425, 450, 475, or 500 mg/m² every two weeks and the antibody that binds to CD47 or antigen-binding fragment thereof is administered to the subject at a dose of 0.3, 1, 2, 4, 8, 15, or 20 mg/kg twice weekly. In certain embodiments, the rituximab is administered to the subject at a dose of 300, 325, 350, 375, 400, 425, 450, 475, or 500 mg/m² every two weeks and the antibody that binds to CD47 or antigen-binding fragment thereof is administered to the subject at a dose of 8 mg/kg twice weekly. In certain embodiments, the rituximab is administered to the subject at a dose of 300, 325, 350, 375, 400, 425, 450, 475, or 500 mg/m² every two weeks and the antibody that binds to CD47 or antigen-binding fragment thereof is administered to the subject at a dose of 8 mg/kg twice weekly. In certain embodiments, the rituximab is administered to the subject at a dose of 300, 325, 350, 375, 400, 425, 450, 475, or 500 mg/m² every two weeks and the antibody that binds to CD47 or antigen-binding fragment thereof is administered to the subject at a dose of 20 mg/kg once weekly.

In certain embodiments, the rituximab anti-CD20 antibody, e.g., rituximab, is administered to the subject at a dose of 300, 325, 350, 375, 400, 425, 450, 475, or 500 mg/m² every four weeks and the antibody that binds to CD47 or antigen-binding fragment thereof is administered to the subject at a dose of 0.3, 1, 2, 4, 8, 15, or 20 mg/kg twice weekly. In certain embodiments, the rituximab is administered to the subject at a dose of 300, 325, 350, 375, 400, 425, 450, 475, or 500 mg/m² every four weeks and the antibody that binds to CD47 or antigen-binding fragment thereof is administered to the subject at a dose of 8 mg/kg twice weekly. In certain embodiments, the rituximab is administered to the subject at a dose of 300, 325, 350, 375, 400, 425, 450, 475, or 500 mg/m² every four weeks and the antibody that binds to CD47 or antigen-binding fragment thereof is administered to the subject at a dose of 8 mg/kg twice weekly. In certain embodiments, the rituximab is administered to the subject at a dose of 300, 325, 350, 375, 400, 425, 450, 475, or 500 mg/m² every four weeks and the antibody that binds to CD47 or antigen-binding fragment thereof is administered to the subject at a dose of 20 mg/kg once weekly.

In certain embodiments, anti-CD20 antibody, e.g., rituximab, is administered to the subject at a dose of 375 mg/m² once weekly and the antibody that binds to CD47 or antigen-binding fragment thereof is administered to the subject at a dose of 0.3, 1, 2, 4, 8, 15, or 20 mg/kg twice weekly. In certain embodiments, rituximab is administered to the subject at a dose of 375 mg/m² once weekly and the antibody that binds to CD47 or antigen-binding fragment thereof is administered to the subject at a dose of 8 mg/kg twice weekly. In certain embodiments, rituximab is administered to the subject at a dose of 375 mg/m² once weekly and the antibody that binds to CD47 or antigen-binding fragment thereof is administered to the subject at a dose of 8 mg/kg twice weekly. In certain embodiments, rituximab is administered to the subject at a dose of 375 mg/m² once weekly and the antibody that binds to CD47 or antigen-binding fragment thereof is administered to the subject at a dose of 20 mg/kg once weekly.

In certain embodiments, anti-CD20 antibody, e.g., rituximab, is administered to the subject at a dose of 375 mg/m² every two weeks and the antibody that binds to CD47 or antigen-binding fragment thereof is administered to the subject at a dose of 0.3, 1, 2, 4, 8, 15, or 20 mg/kg twice weekly. In certain embodiments, rituximab is administered to the subject at a dose of 375 mg/m² every two weeks and the antibody that binds to CD47 or antigen-binding fragment thereof is administered to the subject at a dose of 8 mg/kg twice weekly. In certain embodiments, rituximab is administered to the subject at a dose of 375 mg/m² every two weeks and the antibody that binds to CD47 or antigen-binding fragment thereof is administered to the subject at a dose of 8 mg/kg twice weekly. In certain embodiments, rituximab is administered to the subject at a dose of 375 mg/m² every two weeks and the antibody that binds to CD47 or antigen-binding fragment thereof is administered to the subject at a dose of 20 mg/kg once weekly.

In certain embodiments, anti-CD20 antibody, e.g., rituximab, is administered to the subject at a dose of 375 mg/m² every four weeks and the antibody that binds to CD47 or antigen-binding fragment thereof is administered to the subject at a dose of 0.3, 1, 2, 4, 8, 15, or 20 mg/kg twice weekly. In certain embodiments, rituximab is administered to the subject at a dose of 375 mg/m² every four weeks and the antibody that binds to CD47 or antigen-binding fragment thereof is administered to the subject at a dose of 8 mg/kg twice weekly. In certain embodiments, rituximab is administered to the subject at a dose of 375 mg/m² every four weeks and the antibody that binds to CD47 or antigen-binding fragment thereof is administered to the subject at a dose of 8 mg/kg twice weekly. In certain embodiments, rituximab is administered to the subject at a dose of 375 mg/m² every four weeks and the antibody that binds to CD47 or antigen-binding fragment thereof is administered to the subject at a dose of 20 mg/kg once weekly.

As used herein, the term “in combination” in the context of the administration of other therapies refers to the use of more than one therapy. The use of the term “in combination” does not restrict the order in which therapies are administered. The therapies may be administered, e.g., serially, sequentially, concurrently, or concomitantly.

In certain embodiments of the methods provided herein, an anti-CD20 antibody, e.g., rituximab, is administered prior to a protein therapeutic, for example, an anti-CD47 antibody. In certain embodiments of the methods provided herein, an anti-CD20 antibody, e.g., rituximab, is administered concurrently with a protein therapeutic, for example, an anti-CD47 antibody. In certain embodiments of the methods provided herein, an anti-CD20 antibody, e.g., rituximab, is administered after a protein therapeutic, for example, an anti-CD47 antibody. In certain embodiments of the methods provided herein, an anti-CD20 antibody, e.g., rituximab, is administered prior to and concurrently with a protein therapeutic, for example, an anti-CD47 antibody.

As used herein, the terms “subject” and “patient” are used interchangeably. As used herein, a subject is a mammal such as a non-primate (e.g., cows, pigs, horses, cats, dogs, goats, rabbits, rats, mice, etc.) or a primate (e.g., monkey and human), for example a human. In one embodiment, the subject is a mammal, e.g., a human, diagnosed with a condition or disorder provided herein (e.g., cancer, metastasis, or angiogenesis). In another embodiment, the subject is a mammal, e.g., a human, at risk of developing a condition or disorder provided herein (e.g., cancer, metastasis, or angiogenesis). In another embodiment, the subject is human.

As used herein, “hematological cancer” refers to a cancer of the blood, and includes leukemia, lymphoma and myeloma among others. “Leukemia” refers to a cancer of the blood in which too many white blood cells that are ineffective in fighting infection are made, thus crowding out the other parts that make up the blood, such as platelets and red blood cells. It is understood that cases of leukemia are classified as acute or chronic. Certain forms of leukemia include, by way of non-limiting example, acute lymphocytic leukemia (ALL); acute myeloid leukemia (AML); chronic lymphocytic leukemia (CLL); chronic myelogenous leukemia (CML); Myeloproliferative disorder/neoplasm (MPDS); and myelodysplasia syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma. In certain embodiments, the hematological cancer is multiple myeloma. In certain embodiments, the hematological cancer is non-Hodgkin's lymphoma.

Non-limiting examples of a condition which can be treated or managed with an anti-CD47 antibody described herein, alone or in combination with an anti-CD20 antibody, e.g., rituximab, include hematological cancer and/or solid tumors, as well as diseases or disorders related to aberrant CD47 expression, activity and/or signaling include, by way of non-limiting example, hematological cancer and/or solid tumors. Hematological cancers include, e.g., leukemia, lymphoma and myeloma. Certain forms of leukemia include, by way of non-limiting example, acute lymphocytic leukemia (ALL); acute myeloid leukemia (AML); chronic lymphocytic leukemia (CLL); chronic myelogenous leukemia (CML); Myeloproliferative disorder/neoplasm (MPDS); and myelodysplasia syndrome. Certain forms of lymphoma include, by way of non-limiting example, Hodgkin's lymphoma, both indolent and aggressive non-Hodgkin's lymphoma, Burkitt's lymphoma, and follicular lymphoma (small cell and large cell). Certain forms of myeloma include, by way of non-limiting example, multiple myeloma (MM), giant cell myeloma, heavy-chain myeloma, and light chain or Bence-Jones myeloma. Solid tumors include, e.g., breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors.

Symptoms associated with cancers and other neoplastic disorders include, for example, inflammation, fever, general malaise, fever, pain, often localized to the inflamed area, loss of appetite, weight loss, edema, headache, fatigue, rash, anemia, muscle weakness, muscle fatigue and abdominal symptoms such as, for example, abdominal pain, diarrhea or constipation.

In specific aspects, provided herein are anti-CD47 antibodies useful, alone or in combination with an anti-CD20 antibody, e.g., rituximab, in treating, delaying the progression of, impeding, preventing relapse of or alleviating a symptom of a cancer (e.g., MM, NHL, AML, breast cancer (e.g., triple negative breast cancer), bladder cancer, non-small cell lung cancer/carcinoma, hepatocellular carcinoma (HCC), sarcoma, and head and neck cancer). For example, the CD47 antibodies described herein are useful, alone or in combination with an anti-CD20 antibody, e.g., rituximab, in treating hematological malignancies and/or tumors, e.g., hematological malignancies and/or tumors. For example, the CD47 antibodies described herein are useful in treating CD47+ tumors. By way of non-limiting example, the CD47 antibodies described herein are useful in treating non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), breast cancer (e.g., triple negative breast cancer), ovarian cancer, head and neck cancer, bladder cancer, melanoma, colorectal cancer, pancreatic cancer, lung cancer, leiomyoma, leiomyosarcoma, glioma, glioblastoma, and so on. Solid tumors include, e.g., breast tumors, ovarian tumors, lung tumors (e.g., NSCLC), pancreatic tumors, prostate tumors, melanoma tumors, colorectal tumors, lung tumors, head and neck tumors, bladder tumors, esophageal tumors, liver tumors (e.g., hepatocellular carcinoma), sarcoma, and kidney tumors.

In a specific embodiment, provided herein is a method of treating cancer (e.g., a hematological disorder/cancer or solid cancer) in a subject comprising administering (e.g., administering concurrently or sequentially) to a subject in need thereof (i) an anti-CD47 antibody described herein or antigen-binding fragment thereof which specifically binds to CD47 such as human CD47, alone or in combination with an anti-CD20 antibody, e.g., rituximab, and (ii) another anti-cancer agent. In certain embodiments, the anti-cancer agent is a chemotherapeutic agent (e.g., microtubule disassembly blocker, antimetabolite, topisomerase inhibitor, and DNA crosslinker or damaging agent). In certain embodiments, the anti-cancer agent is a tyrosine kinase inhibitor (e.g., GLEEVEC® (imatinib mesylate) or SUTENT® (SU11248 or Sunitinib)). Other non-limiting examples of tyrosine kinase inhibitors include 706 and AMNI07 (nilotinib). RAD00I, PKC412, gefitinib (IRESSA™), erlotinib (TARCEVA®), sorafenib (NEXAVAR®), pazopanib (VOTRIENT™), axitinib, bosutinib, cediranib (RECENTIN®), SPRYCEL® (dasatinib), lapatinib (TYKERB®), lestaurtinib, neratinib, nilotinib (TASIGNA®), semaxanib, toceranib (PALLADIA™) vandetanib (ZACTIMA™), and vatalanib.

In a specific aspect, provided herein is a method of treating cancer (e.g., a hematological disorder/cancer or solid cancer) in a subject comprising administering (e.g., administering concurrently or sequentially) to a subject in need thereof (i) an anti-CD47 antibody described herein or antigen-binding fragment thereof which specifically binds to CD47 such as human CD47, alone or in combination with an anti-CD20 antibody, e.g., rituximab, and (ii) radiation therapy.

In a particular aspect, provided herein is a method of promoting (e.g., inducing or increasing) phagocytosis, e.g., macrophage mediated phagocytic killing of tumor cells, comprising contacting an effective amount of an anti-CD47 antibody described herein which specifically binds to human CD47 with tumor cells, alone or in combination with an anti-CD20 antibody, e.g., rituximab Also provided herein is a method of promoting (e.g., inducing or increasing) phagocytosis, e.g., macrophage mediated phagocytic killing of tumor cells, in a subject in need thereof (e.g., a subject with tumor cells, such as tumor cells expressing CD47), comprising administering to the subject an effective amount of an anti-CD47 antibody described herein which specifically binds to human CD47, alone or in combination with an anti-CD20 antibody, e.g., rituximab.

In a particular aspect, provided herein is a method of reducing tumor volume, comprising contacting an effective amount of an anti-CD47 antibody described herein which specifically binds to human CD47 with the tumor, alone or in combination withan anti-CD20 antibody, e.g., rituximab Also provided herein is a method of reducing tumor volume in a subject in need thereof (e.g., a subject with a tumor, such as a CD47 expressing tumor), comprising administering to the subject an effective amount of an anti-CD47 antibody described herein which specifically binds to human CD47, alone or in combination with an anti-CD20 antibody, e.g., rituximab.

In a particular aspect, provided herein is a method of inhibiting cancer cell growth or proliferation, comprising contacting an effective amount of an anti-CD47 antibody described herein which specifically binds to human CD47 with cancer cells, alone or in combination with an anti-CD20 antibody, e.g., rituximab. Also provided herein is a method of inhibiting cancer cell growth or proliferation in a subject in need thereof (e.g., a subject with cancer cells, such as CD47 expressing cancer cells), comprising administering to the subject an effective amount of an anti-CD47 antibody described herein which specifically binds to human CD47, alone or in combination with an anti-CD20 antibody, e.g., rituximab.

### 4.2 Compositions

### 4.2.1 Protein Therapeutics

The protein therapeutics used in the methods described herein, for example, in combination with an anti-CD20 antibody, e.g., rituximab, may be any protein therapeutics known to one of skill in the art. In certain embodiments, the protein therapeutic is a cytokine. In certain embodiments, the cytokine is bone morphogenetic protein (BMP), erythropoietin (EPO), granulocyte colony-stimulating factor (G-CSF), granulocyte macrophage colony-stimulating factor (GM-CSF), interferon alpha (IFN-α), interferon beta (IFN-β, interleukin 2 (IL-2), interleukin 11 (IL-11), or interferon gamma (IFN-γ). In certain embodiments, the protein therapeutic is an interleukin. In certain embodiments, the protein therapeutic is not an enzyme.

In certain embodiments, the protein therapeutic is a fusion protein, for example, an Fc-containing fusion protein, e.g., a soluble receptor fusion protein.

### 4.2.2 Antibodies

The protein therapeutics used in the methods described herein, for example, in combination with an anti-CD20 antibody, e.g., rituximab, may be antibody therapeutics. In some embodiments, the anti-CD20 antibody is rituximab. Rituximab (RITUXAN®, Biogen/Genentech) is chimeric murine/human monoclonal IgG₁ kappa antibody directed against the CD20 antigen. Rituximab has an approximate molecular weight of 145 kD and a binding affinity for the CD20 antigen of approximately 8.0 nM. Rituximab is produced by mammalian cell (Chinese Hamster Ovary) suspension culture in a nutrient medium containing the antibiotic gentamicin. Gentamicin is not detectable in the final product. RITUXAN® is a sterile, clear, colorless, preservative-free liquid concentrate for intravenous administration. RITUXAN® is supplied at a concentration of 10 mg/mL in either 100 mg/10 mL or 500 mg/50 mL single-use vials. The product is formulated in polysorbate 80 (0.7 mg/mL), sodium chloride (9 mg/mL), sodium citrate dihydrate (7.35 mg/mL), and Water for Injection. The pH is 6.5. In some embodiments, the anti-CD20 antibody is ocrelizumab. In some embodiments, the the anti-CD20 antibody is ofatumumab. In some embodiments, the the anti-CD20 antibody is obinutuzumab. In some embodiments, the anti-CD20 antibody is tositumomab. In some embodiments, the anti-CD20 antibody is ibritumomab. In some embodiments, the anti-CD20 antibody is ocaratuzumab. In some embodiments, the anti-CD20 antibody is veltuzumab.

As used herein and unless otherwise specified, the terms “about” or “approximately” mean within plus or minus 10% of a given value or range. In instances where an integer is required, the terms mean within plus or minus 10% of a given value or range, rounded either up or down to the nearest integer.

As used herein, the terms “antibody” and “immunoglobulin” and “Ig” are terms of art and can be used interchangeably herein and refer to a molecule with an antigen binding site that specifically binds an antigen.

The antibodies provided herein can include, for example, monoclonal antibodies, recombinantly produced antibodies, monospecific antibodies, multispecific antibodies (including bispecific antibodies), human antibodies, humanized antibodies, murine antibodies (e.g., mouse or rat antibodies), chimeric antibodies, synthetic antibodies, and tetrameric antibodies comprising two heavy chain and two light chain molecules. In specific embodiments, antibodies can include, but are not limited to an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, and monovalent antibodies. In a specific embodiment, antibodies can include antigen-binding fragments or epitope binding fragments such as, but not limited to, single chain antibodies or single-chain Fvs (scFv) (e.g., including monospecific, bispecific, etc.), camelized antibodies, affybodies, Fab fragments, F(ab′) fragments, F(ab′)₂ fragments, and disulfide-linked Fvs (sdFv). In certain embodiments, antibodies described herein refer to polyclonal antibody populations.

Antibodies can be of any type (e.g., IgG, IgE, IgM, IgD, IgA or IgY), any class, (e.g., IgG₁, IgG₂, IgG₃, IgG₄, IgA₁ or IgA₂), or any subclass (e.g., IgG2a. or IgG2b) of immunoglobulin molecule. In certain embodiments, antibodies described herein are IgG antibodies, or a class (e.g., human IgG₁, IgG₂, IgG₃ or IgG₄) or subclass thereof. In certain embodiments, antibodies described herein are IgGi antibodies (e.g., human IgG₁) or a subclass thereof. In certain embodiments, IgGi antibodies described herein comprise one or more amino acid substitutions and/or deletions in the constant region. In certain embodiments, antibodies described herein are IgG₄ antibodies (e.g., human IgG₄) or a subclass thereof. In certain embodiments, IgG₄ antibodies described herein comprise one or more amino acid substitutions and/or deletions in the constant region.

As used herein, an “antigen” is a moiety or molecule that contains an epitope to which an antibody can specifically bind. As such, an antigen is also specifically bound by an antibody.

As used herein, an “epitope” is a term in the art and refers to a localized region of an antigen to which an antibody can specifically bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure.

As used herein, the term “monoclonal antibody” is a well known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell or cell line wherein the antibody immunospecifically binds to a CD47 epitope as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody.

As used herein, the term “non-natural amino acid” refers to an amino acid that is not a proteinogenic amino acid, or a post-translationally modified variant thereof. In particular, the term refers to an amino acid that is not one of the 20 common amino acids or pyrrolysine or selenocysteine, or post-translationally modified variants thereof.

As used herein, the term “polyclonal antibodies” refers to an antibody population that includes a variety of different antibodies that immunospecifically bind to the same and/or to different epitopes within an antigen or antigens.

As used herein, the terms “variable region” or “variable domain” refer to a portion of an antibody, generally, a portion of an antibody light or heavy chain, typically about the amino-terminal 110 to 120 amino acids in a mature heavy chain and about the amino-terminal 90 to 100 amino acids in a mature light chain. Variable regions comprise complementarity determining regions (CDRs) flanked by framework regions (FRs). Generally, the spacial orientation of CDRs and FRs are as follows, in an N-terminal to C-terminal direction: FR1-CDR1-1-R2-CDR2-FR3-CDR3-FR4. Without wishing to be bound by any particular mechanism or theory, it is believed that the CDRs of the light and heavy chains are primarily responsible for the interaction of the antibody with antigen and for the specificity of the antibody for an epitope. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242. In certain embodiments, the variable region is a human variable region. In certain embodiments, the variable region comprises murine (e.g., mouse or rat) CDRs and human framework regions (FRs). In particular embodiments, the variable region is a primate (e.g., human or non-human primate) variable region. In certain embodiments, the variable region comprises murine (e.g., mouse or rat) CDRs and primate (e.g., human or non-human primate) framework regions (FRs). As a non-limiting example, a variable region described herein is obtained from assembling two or more fragments of human sequences into a composite human sequence.

In certain aspects, the CDRs of an antibody can be determined according to (i) the Kabat numbering system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; and U.S. Patent No. 7,709,226); or (iii) the ImMunoGeneTics (IMGT) numbering system, for example, as described in Lefranc, M.-P., 1999, The Immunologist, 7:132-136 and Lefranc, M.-P. et al., 1999, Nucleic Acids Res., 27:209-212 (“IMGT CDRs”); or (iv) MacCallum et al., 1996, J. Mol. Biol., 262:732-745. See also, e.g., Martin, A., “Protein Sequence and Structure Analysis of Antibody Variable Domains,” in Antibody Engineering, Kontermann and Dilbel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

Antibodies provided herein can be of any type (e.g., IgG, IgE, IgM, IgD, IgA or IgY), any class, (e.g., IgG₁, IgG₂, IgG₃, IgG₄, IgA₁ or IgA₂), or any subclass (e.g., IgG2a. or IgG2b, or a mixture thereof) of immunoglobulin molecule. In certain embodiments, antibodies described herein are IgG antibodies (e.g., human IgG), or a class (e.g., human IgG₁, IgG₂, IgG₃ or IgG₄) or subclass thereof.

In specific aspects, provided herein is an antibody comprising an antibody light chain and heavy chain, e.g., a separate light chain and heavy chain. With respect to the light chain, in a specific embodiment, the light chain of an antibody described herein is a kappa (κ) light chain. In another specific embodiment, the light chain of an antibody described herein is a lambda (λ) light chain. In another embodiment, light chain is a mixed sequence, e.g., the variable portion of the light chain comprises kappa light chain sequences and the constant region of the light chain comprises lambda light chain sequences, or vice versa. In certain embodiments, the light chain of an antibody described herein is a human kappa light chain or a human lambda light chain. Non-limiting examples of human constant region sequences have been described in the art, e.g., see U.S. Pat. No. 5,693,780 and Kabat et al. (1991 Sequences of Proteins of Immunological Interest, Fifth Edition, U.S. Department of Health and Human Services, NIH Publication No. 91-3242.

In certain embodiments, an anti-CD47 antibody described herein or an antigen-binding fragment thereof comprises amino acid sequences with certain percent identity relative to a parental antibody.

The determination of percent identity between two sequences (e.g., amino acid sequences or nucleic acid sequences) can be accomplished using a mathematical algorithm.

A non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm nih gov). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

Anti-CD47 antibodies described herien also include monoclonal antibodies that specifically bind CD47, wherein the antibody does not promote (e.g., induce or increase), or cause a significant level of, agglutination, e.g., red blood cell hemagglutination (“RBC hemagglutination”).

Pharmaceutical compositions according to the invention can include an antibody of the invention and a pharmaceutically acceptable carrier.

In some embodiments, the antibody or antigen-binding fragment thereof is an IgG isotype. In some embodiments, the constant region of the antibody is of human IgG1 isotype, having an amino acid sequence:

(SEQ ID NO: 1)
ASTKGPSVFP LAPSSKSTSG GTAALGCLVK
DYFPEPVTVS WNSGALTSGV HTFPAVLQSS GLYSLSSVVT
VPSSSLGTQT YICNVNHKPS NTKVDKKVEPEYKCKVSNKA
LPAPIEKTIS KAKGQPREPQ VYTLPPSRDE LTKNQVSLTC
LVKGFYPSDI AVEWESNGQP ENNYKTTPPV LDSDGSFFLY
SKLTVDKSRW QQGNVFSCSV MHEALHNHYT QKSLSLSPGK

In some embodiments, the human IgG1 constant region is modified at amino acid Asn297 (Boxed, Kabat Numbering) to prevent to glycosylation of the antibody, for example Asn297Ala (N297A). In some embodiments, the constant region of the antibody is modified at amino acid Leu235 (Kabat Numbering) to alter Fc receptor interactions, for example Leu235Glu (L235E) or Leu235Ala (L235A). In some embodiments, the constant region of the antibody is modified at amino acid Leu234 (Kabat Numbering) to alter Fc receptor interactions, e.g., Leu234Ala (L234A). In some embodiments, the constant region of the antibody is altered at both amino acid 234 and 235, for example Leu234Ala and Leu235Ala (L234A/L235A) (EU index of Kabat et al 1991 Sequences of Proteins of Immunological Interest).

In some embodiments, the constant region of the antibody is of human IgG2 isotype, having an amino acid sequence:

(SEQ ID NO: 2)
ASTKGPSVFP LAPCSRSTSE STAALGCLVK
DYFPEPVTVS WNSGALTSGV HTFPAVLQSS GLYSLSSVVT
VPSSNFGTQT YTCNVDHKPS NTKVDKTVER KCCVECPPCP
APPVAGPSVF LFPPKPKDTL MISRTPEVTC
VVVDVSHEDPKVSNKGLPAP IEKTISKTKG QPREPQVYTL
PPSREEMTKN QVSLTCLVKG FYPSDISVEW ESNGQPENNY
KTTPPMLDSD GSFFLYSKLT VDKSRWQQGN VFSCSVMHEA
LHNHYTQKSL SLSPGK

In some embodiments, the human IgG2 constant region is modified at amino acid Asn297 (Boxed, Kabat Numbering) to prevent to glycosylation of the antibody, e.g., Asn297Ala (N297A).

In some embodiments, the constant region of the antibody is of human IgG3 isotype, having an amino acid sequence:

(SEQ ID NO: 3)
ASTKGPSVFP LAPCSRSTSG GTAALGCLVK DYFPEPVTVS
WNSGALTSGV HTFPAVLQSS GLYSLSSVVT VPSSSLGTQT
YTCNVNHKPS NTKVDKRVEL KTPLGDTTHT CPRCPEPKSC
DTPPPCPRCP EPKSCDTPPP CPRCPEPKSC DTPPPCPRCP
APELLGGPSV FLFPPKPKDT LMISRTPEVT CVVVDVSHED
CKVSNKALPA PIEKTISKTK GQPREPQVYT LPPSREEMTK
NQVSLTCLVK GFYPSDIAVE WESSGQPENN YNTTPPMLDS
DGSFFLYSKL TVDKSRWQQG

In some embodiments, the human IgG3 constant region is modified at amino acid Asn297 (Boxed, Kabat Numbering) to prevent to glycosylation of the antibody, e.g., Asn297Ala (N297A). In some embodiments, the human IgG3 constant region is modified at amino acid 435 to extend the half-life, e.g., Arg435H is (R435H) (EU index of Kabat et al 1991 Sequences of Proteins of Immunological Interest).

In some embodiments, the constant region of the antibody is of human IgG4 isotype, having an amino acid sequence:

(SEQ ID NO: 4)
ASTKGPSVFP LAPCSRSTSE STAALGCLVK
DYFPEPVTVS WNSGALTSGV HTFPAVLQSS GLYSLSSVVT
VPSSSLGTKT YTCNVDHKPS NTKVDKRVES CKVSNKGLPS
SIEKTISKAK GQPREPQVYT LPPSQEEMTK NQVSLTCLVK
GFYPSDIAVE WESNGOPENN YKTTPPVLDS DGSFFLYSRL
TVDKSRWQEG NVFSCSVMHE ALHNHYTQKS LSLSLGK

In some embodiments, the human IgG4 constant region is modified within the hinge region to prevent or reduce strand exchange, e.g., Ser228Pro (S228P). In other embodiments, the human IgG4 constant region is modified at amino acid 235 to alter Fc receptor interactions, e.g., Leu235Glu (L235E). In some embodiments, the human IgG4 constant region is modified within the hinge and at amino acid 235, e.g., Ser228Pro and Leu235Glu (S228P/L235E). In some embodiments, the human IgG4 constant region is modified at amino acid Asn297 (Kabat Numbering) to prevent to glycosylation of the antibody, e.g., Asn297Ala (N297A). In some embodiments of the invention, the human IgG4 constant region is modified at amino acid positions Ser228, Leu235, and Asn297 (e.g., S228P/L235E/N297A). (EU index of Kabat et al 1991 Sequences of Proteins of Immunological Interest). In other embodiments of the invention, the antibody is of human IgG4 subclass and lacks glycosylation. In these embodiments the glycosylation can be eliminated by mutation at position 297 (Kabat numbering), for example N297A. In other embodiments, the glycosylation can be eliminated by production of the antibody in a host cell that lacks the ability for post-translational glycosylation, for example a bacterial or yeast derived system or a modified mammalian cell expression system.

In some embodiments, the human IgG constant region is modified to enhance FcRn binding. Examples of Fc mutations that enhance binding to FcRn are Met252Tyr, Ser254Thr, Thr256Glu (M252Y, S254T, T256E, respectively) (Kabat numbering, Dall'Acqua et al 2006, J. Biol Chem Vol 281 (33) 23514-23524), or Met428Leu and Asn434Ser (M428L, N434S) (Zalevsky et al 2010 Nature Biotech, Vol 28 (2) 157-159). (EU index of Kabat et al 1991 Sequences of Proteins of Immunological Interest).

In some embodiments, the human IgG constant region is modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1):1-11.

In some embodiments, the human IgG constant region is modified to induce heterodimerization. For example, having an amino acid modification within the CH3 domain at Thr366, which when replaced with a more bulky amino acid, e.g., Try (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248: 7-15).

In other embodiments of the invention, the antibody lacks glycosylation, but is not modified at amino acid Asn297 (Kabat numbering). In these embodiments the glycosylation can be eliminated by production of the antibody in a host cell that lacks a post-translational glycosylation capacity, for example a bacterial or yeast derived system or a modified mammalian cell expression system.

### 4.2.2.1 Anti-CD47 Antibodies for Use in the Methods Provided Herein

In a specific aspect, for use in the methods provided herein are antibodies which specifically bind to CD47 (e.g., human CD47). Such anti-CD47 antibodies include all antibodies disclosed in U.S. Patent Application Publication No. 2014/0140989, which is incorporated by reference herein in its entirety. Such anti-CD47 antibodies also include all antibodies disclosed in International Patent Application Publication No. WO 2016/109415, which is incorporated by reference herein in its entirety.

As used herein, the terms “CD47” or “integrin-associated protein” or “IAP” or “ovarian cancer antigen” or “OA3” or “Rh-related antigen” or “MERG” can be used interchangeably and refer to a multi-spanning transmembrane receptor belonging to the immunoglobulin superfamily The amino acid sequence of an exemplary human CD47 is provided below (GenBank Accession No. Q08722.1 (GI:1171879), incorporated herein by reference). The signal sequence (amino acids 1-18) is underlined.

```
(SEQ ID NO: 48)
    1 MWPLVAALLL GSACCGSAQL LENKTKSVEF
        TFCNDTVVIP CFVINMEAQN TTEVYVKWKF
  61 KGRDIYTFDG ALNKSTVPTD FSSAKIEVSQ
        LLKGDASLKM DKSDAVSHTG NYTCEVTELT
121 REGETIIELK YRVVSWFSPN ENILIVIFPI
        FAILLFWGQF GIKTLKYRSG GMDEKTIALL
181 VAGLVITVIV IVGAILFVPG EYSLKNATGL
        GLIVISTGIL ILLHYYVEST AIGLTSFVIA
241 ILVIQVIAYI LAVVGLSLCI AACIPMHGPL
        LISGLSILAL AQLLGLVYMK FVASNQKTIQ
301 PPRKAVEEPL NAFKESKGMM NDE
```

For clarity, the amino acid sequence of an exemplary human CD47 excluding the signal seauence is provided below.

```
(SEQ ID NO: 49)
    1 QLLFNKTKSV EFTFCNDTVV IPCFVINMEA
        QNTTEVYVKW KFKGRDIYTF DGALNKSTVP
  61 TDESSAKIEV SQLLKGDASL KMDKSDAVSH
        TGNYTCEVTE LTREGETIIE LKYRVVSWFS
121 PNENILIVIF PIFAILLFWG QFGIKTLKYR
        SGGMDEKTIA LLVAGLVITV IVIVGAILFV
181 PGEYSLKNAT GLGLIVTSTG ILILLHYYVF
        STAIGLTSFV IAILVIQVIA YILAVVGLSL
241 CIAACIPMHG PLLISGLSIL ALAQLLGLVY
        MKFVASNQKT IQPPRKAVEE PLNAFKESKG
301 MMNDE
```

The invention also provides pharmaceutical compositions that include one or more monoclonal antibodies that bind to CD47 or an immunologically active fragment thereof, wherein the antibody does not cause a significant level of hemagglutination of red blood cells after administration.

Hemagglutination is an example of a homotypic interaction, wherein two CD47 expressing cells are caused to aggregate or clump when treated with a bivalent CD47 binding entity. The ability of the antibodies of the present invention to bind CD47 on the cell surface and not cause a cellular clumping phenomenon is not limited to red blood cells. The antibodies of the present invention have been observed to uniquely bind CD47 in a manner that does not promote clumping of CD47 positive cell lines, e.g., Daudi cells.

In some cases, the antibody for use in the methods provided herein comprises a variable heavy (VH) chain region selected from the group consisting of SEQ ID NOs: 5-30. The antibody optionally comprises a variable light (VL) chain region selected from the group consisting of SEQ ID NOs: 31-47. In some cases, the antibody comprises a VH chain region selected from the group consisting of SEQ ID NOs: 5-30 and a VL chain region selected from the group consisting of SEQ ID NOs: 31-47. The antibodies of the invention also include antibodies having a variable heavy chain that is at least 90%, 91%, 92%, 93%, 94%, 95%, 96%, 97%, 98%, 99% or more identical to the sequence set forth in at least one of SEQ ID NOs: 5-30 and a variable light chain that is at least 90%, 91%, 92%, 93%, 94%, 95%, 96%, 97%, 98%, 99% or more identical to the sequence set forth in at least one of SEQ ID NOs: 31-47. In other aspects, the antibody comprises a VH region provided in any one of SEQ ID NOs: 5, 7, 8, 11, 15-17, 20-22, and 27-30 paired with a VL region provided in any one of SEQ ID NOs: 31-39, 42, 43, 44, and 47. In another embodiment, the antibody comprises a VH region provided in any one of SEQ ID NOs: 5, 7, 8, 11, 12, 15-17, 20-22, and 27-30 paired with a VL region provided in any one of SEQ ID NOs: 31, 32, 35, 40, 41, 42, 43, 44, and 47. In yet another aspect, the antibody comprises a combination of a VH chain region and a VL chain region selected from the combinations listed in Table 1.

TABLE 1
Exemplary anti-CD47 antibody VH/VL combinations.

| Antibody | Variable heavy (VH) chain | Variable light (VL) chain |
|---|---|---|
| 2A1 | SEQ ID NO: 5 | SEQ ID NO: 31 |
| 2A1-xi | SEQ ID NO: 5 | SEQ ID NO: 32 |
| AB2.03 | SEQ ID NO: 7 | SEQ ID NO: 33 |
| AB2.04 | SEQ ID NO: 7 | SEQ ID NO: 34 |
| AB2.05 | SEQ ID NO: 7 | SEQ ID NO: 35 |
| AB2.06 | SEQ ID NO: 7 | SEQ ID NO: 36 |
| AB2.07 | SEQ ID NO: 7 | SEQ ID NO: 37 |
| AB2.08 | SEQ ID NO: 7 | SEQ ID NO: 38 |
| AB2.09 | SEQ ID NO: 7 | SEQ ID NO: 39 |
| AB2.13 | SEQ ID NO: 7 | SEQ ID NO: 43 |
| AB3.09 | SEQ ID NO: 8 | SEQ ID NO: 39 |
| AB6.12 | SEQ ID NO: 11 | SEQ ID NO: 42 |
| AB6.13 | SEQ ID NO: 11 | SEQ ID NO: 43 |
| AB6.14 | SEQ ID NO: 11 | SEQ ID NO: 44 |
| AB6.17 | SEQ ID NO: 11 | SEQ ID NO: 47 |
| AB10.13 | SEQ ID NO: 15 | SEQ ID NO: 43 |
| AB10.14 | SEQ ID NO: 15 | SEQ ID NO: 44 |
| AB11.05 | SEQ ID NO: 16 | SEQ ID NO: 35 |
| AB12.05 | SEQ ID NO: 17 | SEQ ID NO: 35 |
| AB15.05 | SEQ ID NO: 20 | SEQ ID NO: 35 |
| AB16.05 | SEQ ID NO: 21 | SEQ ID NO: 35 |
| AB17.05 | SEQ ID NO: 22 | SEQ ID NO: 35 |
| AB22.05 | SEQ ID NO: 27 | SEQ ID NO: 35 |
| AB23.05 | SEQ ID NO: 28 | SEQ ID NO: 35 |
| AB24.05 | SEQ ID NO: 29 | SEQ ID NO: 35 |
| AB25.05 | SEQ ID NO: 30 | SEQ ID NO: 35 |

In some embodiments, the CD47 antibody or antigen-binding fragment thereof for use in the methods provided herein comprises a VH complementarity determining region 1 (CDR1) sequence set forth in SEQ ID NO: 50, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, or SEQ ID NO: 66, a VH CDR2 sequence set forth in SEQ ID NO: 51, SEQ ID NO: 72, SEQ ID NO: 73, SEQ ID NO: 74, SEQ ID NO: 75, or SEQ ID NO: 76, a VH CDR3 sequence set forth in SEQ ID NO: 52 or SEQ ID NO: 77, a VL CDR1 sequence set forth in SEQ ID NO: 53, SEQ ID NO: 67, or SEQ ID NO: 68, a VL CDR2 sequence set forth in SEQ ID NO: 54, SEQ ID NO: 69, SEQ ID NO: 70, or SEQ ID NO: 71 and a VL CDR3 sequence set forth in SEQ ID NO: 55. For example, the antibody or immunologically active fragment thereof comprises a VH CDR1 sequence set forth in SEQ ID NO: 50, a VH CDR2 sequence set forth in SEQ ID NO: 51, a VH CDR3 sequence set forth in SEQ ID NO: 52, a VL CDR1 sequence set forth in SEQ ID NO: 53, a VL CDR2 sequence set forth in SEQ ID NO: 54, and a VL CDR3 sequence set forth in SEQ ID NO: 55. In another example, the antibody or immunologically active fragment thereof comprises a VH CDR1 sequence set forth in SEQ ID NO: 50, a VH CDR2 sequence set forth in SEQ ID NO: 72, a VH CDR3 sequence set forth in SEQ ID NO: 52, a VL CDR1 set forth in SEQ ID NO: 53, a VL CDR2 sequence set forth in SEQ ID NO: 71, and a VL CDR3 sequence set forth in SEQ ID NO: 55.

In a specific aspect, provided herein is an antibody, e.g. a monoclonal antibody, which specifically binds to human CD47, wherein such an anti-CD47 antibody is a variant of a parental anti-CD47 antibody, wherein the anti-CD47 antibody, when produced using a cell-free (CF) expression system, has a higher antibody expression titer or yield compared to that of the parental anti-CD47 antibody when expressed in the CF system, and wherein the anti-CD47 antibody comprises one or more amino acid modifications, for example, 1-15 amino acid modifications, relative to the the parental anti-CD47 antibody. In a particular aspect, the one or more amino acid modifications, for example, 1-15 amino acid modifications, are within the heavy chain or VH (e.g., SEQ ID NO: 6). In a particular aspect, the one or more amino acid modifications, for example, 1-15 amino acid modifications, are within the framework region of a VH (e.g., SEQ ID NO: 6). In a certain aspect, the anti-CD47 antibody provided herein which is a variant of a parental anti-CD47 antibody comprising the CDRs (e.g., Kabat CDRs) of the parental anti-CD47 antibody.

In a specific aspect, provided herein is an antibody, e.g. a monoclonal antibody, which specifically binds to human CD47, wherein such an anti-CD47 antibody is a variant of a parental anti-CD47 antibody, wherein the anti-CD47 antibody, when produced using a cell-free (CF) expression system, has a higher antibody expression titer or yield compared to that of the parental anti-CD47 antibody when expressed in the CF system, and wherein the anti-CD47 antibody comprising one or more amino acid modifications, for example, 1-15 amino acid modifications, relative to the the parental anti-CD47 antibody. In a particular aspect, the one or more amino acid modifications, for example, 5 or 14 amino acid modifications, are within the heavy chain or VH (e.g., SEQ ID NO: 6). In a particular aspect, the one or more amino acid modifications, for example, 5, 10, 13 or 14 amino acid modifications, are within the framework region of a VH (e.g., SEQ ID NO: 6). In a particular aspect, the one or more amino acid modifications, for example, 5, 13 or 14 amino acid modifications are within the framework region of a VH (e.g., SEQ ID NO: 6). In a certain aspect, the anti-CD47 antibody provided herein which is a variant of a parental anti-CD47 antibody comprising the CDRs (e.g., Kabat CDRs) of the parental anti-CD47 antibody. In certain aspects, such anti-CD47 antibody is an IgG1, IgG2, IgG3, or IgG4 isotype antibody. In certain aspects, such anti-CD47 antibody is an IgG1 isotype antibody. In certain aspects, such anti-CD47 antibody is an IgG1 Z allotype isotype antibody. In certain aspects, such anti-CD47 antibody is an IgG4, such as an IgG4P or IgG4PE, isotype antibody.

In a specific aspect, provided herein is an antibody, e.g. a monoclonal antibody, which specifically binds to human CD47, wherein such an anti-CD47 antibody is a variant of a parental anti-CD47 antibody, wherein the anti-CD47 antibody, when produced using a cell-free (CF) expression system, has a higher antibody expression titer or yield compared to that of the parental anti-CD47 antibody when expressed in the CF system. In specific embodiments, the parental anti-CD47 antibody is antibody AB6.12 (see, e.g., U.S. Application Publication No. US 2014/0140989 A1, which is incorporated herein by reference in its entirety). The amino acid sequences of the heavy chain variable region (VH) and light chain variable region (VL) of antibody AB6.12 are provided below, wherein the Kabat CDRs are underlined. In a certain aspect, the anti-CD47 antibody provided herein is a variant of parental antibody AB6.12, and comprises the CDRs (e.g., Kabat CDRs) of parental antibody AB6.12, for example SEQ ID NOs: 50, 72, 52, 53, 71, and 55. In certain aspects, such anti-CD47 antibody is an IgG1, IgG2, IgG3, or IgG4 isotype antibody. In certain aspects, such anti-CD47 antibody is an IgG1 isotype antibody. In certain aspects, such anti-CD47 antibody is an IgG1 Z allotype isotype antibody. In certain aspects, such anti-CD47 antibody is an IgG4, such as an IgG4P or IgG4PE, isotype antibody.

Anti-CD47 antibody AB6.12 heavy chain variable region (VH) (Kabat CDRs 1-3 are underlined, SEQ ID NOs: 50, 72, and 52):

(SEQ ID NO: 6)
QMQLVQSGAEVKKTGSSVKVSCKASGFNIKDYYLHWV
RQAPGQALEWMGWIDPDQGDTEYAQKFQDRVTITRDR
SMSTAYMELSSLRSEDTAMYYCNAAYGSSSYPMDYWG
QGTTVTV

Anti-CD47 antibody AB6.12 light chain variable region (VL) (Kabat CDRs 1-3 are underlined, SEQ ID NOs: 53, 71, and 55):

(SEQ ID NO: 42)
NIQMTQSPSAMSASVGDRVTITCKASQDIHRYLSWF
QQKPGKVPKHLIYRANRLVSGVPSRFSGSGSGTEFT
LTISSLQPEDFATYYCLQYDEFPYTFGGGTKVEIK

In a specific embodiment, an anti-CD47 described herein comprises one or more amino acid modifications (e.g., 1-15 amino acid modifications), for example in the VH framework region, of a parental antibody, e.g., a parental antibody selected from anti-CD47 antibodies described herein.

In a specific aspect, provided herein is an antibody, e.g. a monoclonal antibody, which specifically binds to human CD47, wherein such an anti-CD47 antibody is a variant of a parental anti-CD47 antibody, wherein the anti-CD47 antibody, when produced using a cell-free (CF) expression system, has a higher antibody expression titer or yield compared to that of the parental anti-CD47 antibody when expressed in the CF system, and wherein the anti-CD47 antibody comprises a VH comprising the following N-terminal to C-terminal sequence: X₁QL₂QLVQSGAEVKKX₃GX₄SVKVSCKASGFNIKDYYLHWVRQAPGQX₅LEWMGW IDPDQGDTEYAQKX₆QX₇RVTX₈TX₉DX₁₀SX₁₁STAYMELX₁₂SLRSX₁₃DTAX₁₄YYCNA AYGSSSYPMDYWGQGTTVTV (SEQ ID NO: 89), wherein the underlined amino acid residues for X₁-X₁₄ are ordered from N-terminus to C-terminus, wherein Xi is M or there is no amino acid at position X₁, X₂ is an amino acid with hydrophobic side chains such as M or V, X₃ is T or P, X₄ is S or A, X₅ is an acid having aliphatic side chains such as A or G, X₆ is F or L, X₇ is D or G, X₈ is an amino acid with hydrophobic side chains such as I or M, X₉ is R or T, X₁₀ is R or T, X₁₁ is M or T, X₁₂ is S or R, X₁₃ is a negatively charged amino acid such as E or D, and X₁₄ is an amino acid with hydrophobic side chains such as M or V.

In particular aspects, an anti-CD47 antibody described herein comprises a VH comprising the sequence of SEQ ID NO: 89, wherein the amino acid at position X₁ is any amino acid such as M, X₂ is not M, X₃ is not T, X₄ is not S, X₅ is not A, X₆ is not F, X₇ is not D, X₈ is not I, X₉ is not R, X₁₀ is not R, X₁₁ is not M, X₁₂ is not S, X₁₃ is not E, and/or X₁₄ is not M. In particular aspects, any 1, 2, 3, 4, 5, 6, 7, 8, 9, 10, 11, 12, 13, or 14 of X₁ to X₁₄ are not these amino acids. In particular aspects, the VH amino acid sequence is not the VH amino acid sequence of antibody AB6.12, for example, the VH amino acid sequence is not SEQ ID NO: 6.

In particular aspects, an anti-CD47 antibody described herein comprises a VH comprising the sequence of SEQ ID NO: 89, wherein the amino acid at position X₇ is not G, X₉ is not A and/or Xii is not S. In particular aspects, any 1, 2, or 3 of X₇, X₉ and X₁₁ are not these amino acids. In particular aspects, when the amino acid at position X₇ is G, then X₈ is M and/or X₁₀ is T, X₉ is not A and/or X₁₁ is not S.

In particular aspects, an anti-CD47 antibody described herein comprises a VH comprising the sequence of SEQ ID NO: 89, wherein the amino acid at position X₇ is not G, X₈ is not M, X₉ is not E, X₁₀ is not T, and/or Xii is not T. In particular aspects, any 1, 2, 3, or 4 of X₇ to X₁₁ are not these amino acids. In particular aspects, when the amino acid at position X₇ is G, then X₈ is M, X₁₀ is T, X₉ is not E, and X₁₁ is T.

In a particular aspect, an anti-CD47 antibody described herein comprises a VH comprising the sequence of SEQ ID NO: 89, wherein the VH does not comprise the amino acid sequence of SEQ ID NO: 5, 6, 7, 8, 9, 10, 12, 13, 14, 15, 16, 17, 18, 19, 20, 21, 22, 23, 24, 25, 26, 27, 28, 29, or 30, of U.S. Application Publication No. US2014/0140989 A1, which is incorporated herein by reference in its entirety. In a particular aspect, an anti-CD47 antibody described herein comprises a VH comprising the consensus sequence of SEQ ID NO: 89, wherein the VH does not comprise the framework regions of the amino acid sequence of SEQ ID NO: 5, 6, 7, 8, 9, 10, 12, 13, 14, 15, 16, 17, 18, 19, 20, 21, 22, 23, 24, 25, 26, 27, 28, 29, or 30, of U.S. Application Publication No. US2014/0140989 A1, which is incorporated herein by reference in its entirety.

In particular aspects, X₁ is M, X₂ is V, X₃ is P, X₄ is A, X₅ is G, X₆ is L, X₇ is G, X₈ is M, X₉ is T, X₁₀ is T, X₁₁ is T, X₁₂ is R, X₁₃ is D, and/or X₁₄ is V. In particular embodiments, any 1, 2, 3, 4, 5, 6, 7, 8, 9, 10, 11, 12, 13, or 14 of X₁ to X₁₄ are these amino acids.

In particular aspects, X₁ is M, X₂ is M, X₃ is P, X₄ is 5, X₅ is A, X₆ is F, X₇ is G, X₈ is I, X₉ is R, X₁₀ is R, X₁₁ is T, X₁₂ is R, X₁₃ is E, and/or X₁₄ is V. In particular embodiments, any 1, 2, 3, 4, 5, 6, 7, 8, 9, 10, 11, 12, 13, or 14 of X₁ to X₁₄ are these amino acids.

In a particular aspect, an anti-CD47 antibody provided herein is not antibody AB6.12. In a particular aspect, an anti-CD47 antibody provided herein does not comprise a VH (e.g., SEQ ID NO: 6) and/or a VL (e.g., SEQ ID NO: 42) of antibody AB6.12.

In a specific aspect, an anti-CD47 antibody provided herein, comprises one of the following VH amino acid sequences presented in Table 2.

TABLE 2
VH amino acid sequence

| SEQ ID | | |
|---|---|---|
| NO: | Description | VH amino acid sequence |
| 89 | Consensus | X₁QX₂QLVQSGAEVKKX₃GX₄SVK |
| | | VSCKASGFNIKDYYLHWVRQAPGQ |
| | | X₅LEWMGWIDPDQGDTEYAQKX₆Q |
| | | X₇RVTX₈TX₉DX₁₀SX₁₁STAYME |
| | | LX₁₂SLRSX₁₃DTAX₁₄YYCNAAY |
| | | GSSSYPMDYWGQGTTVTV |
| 90 | 13m | MQVQLVQSGAEVKKPGASVKVSCK |
| | | ASGFNIKDYYLHWVRQAPGQGLEW |
| | | MGWIDPDQGDTEYAQKLQGRVTMT |
| | | TDTSTSTAYMELRSLRSDDTAVYY |
| | | CNAAYGSSSYPMDYWGQGTTVTV |
| 91 | 5m | MQMQLVQSGAEVKKPGSSVKVSCK |
| | | ASGFNIKDYYLHWVRQAPGQALEW |
| | | MGWIDPDQGDTEYAQKLQGRVTMT |
| | | TDTSTSTAYMELRSLRSDDTAVYY |
| | | CNAAYGSSSYPMDYWGQGTTVTV |

In a specific aspect, provided herein is an antibody, e.g. a monoclonal antibody, which specifically binds to human CD47, wherein such an anti-CD47 antibody is a variant of a parental anti-CD47 antibody, wherein the anti-CD47 antibody, when produced using a cell-free (CF) expression system, has a higher antibody expression titer or yield compared to that of the parental anti-CD47 antibody when expressed in the CF system, and wherein the anti-CD47 antibody comprises a VH comprising SEQ ID NO: 90. In certain aspects, such anti-CD47 antibody is an IgG1, IgG2, IgG3, or IgG4 isotype antibody. In certain aspects, such anti-CD47 antibody is an IgG1 isotype antibody. In certain aspects, such anti-CD47 antibody is an IgG1 Z allotype isotype antibody. In certain aspects, such anti-CD47 antibody is an IgG4, such as an IgG4P or IgG4PE, isotype antibody.

In a specific aspect, provided herein is an antibody, e.g. a monoclonal antibody, which specifically binds to human CD47, wherein such an anti-CD47 antibody is a variant of a parental anti-CD47 antibody, wherein the anti-CD47 antibody, when produced using a cell-free (CF) expression system, has a higher antibody expression titer or yield compared to that of the parental anti-CD47 antibody when expressed in the CF system, and wherein the anti-CD47 antibody comprises a VH comprising SEQ ID NO: 91. In certain aspects, such anti-CD47 antibody is an IgG1, IgG2, IgG3, or IgG4 isotype antibody. In certain aspects, such anti-CD47 antibody is an IgG1 isotype antibody. In certain aspects, such anti-CD47 antibody is an IgG1 Z allotype isotype antibody. In certain aspects, such anti-CD47 antibody is an IgG4, such as an IgG4P or IgG4PE, isotype antibody.

In a particular aspect, an anti-CD47 antibody (IgG1-13m) provided herein comprises an IgG1 heavy chain comprising the amino acid sequence as set forth below:

(SEQ ID NO: 81)
MQVQLVQSGAEVKKPGASVKVSCKASGFNIKDYYLHWVRQ
APGQGLEWMGWIDPDQGDTEYAQKLQGRVTMTTDTSTSTA
YMELRSLRSDDTAVYYCNAAYGSSSYPMDYWGQGTTVTVS
SASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDKTHTCPPCPAPELLG
GPSVFLFPPKPKDTLMISRTPEVTCVVVDVSHEDPEVKEN
WYVDGVEVHNAKTKPREEQYNSTYRVVSVLTVLHQDWLNG
KEYKCKVSNKALPAPIEKTISKAKGQPREPQVYTLPPSRD
ELTKNQVSLICLVKGFYPSDIAVEWESNGQPENNYKTTPP
VLDSDGSFFLYSKLTVDKSRWQQGNVFSCSVMHEALHNHY
TQKSLSLSPGK

In a particular aspect, an anti-CD47 antibody (IgG1-13mZ) provided herein comprises an IgG1-Z allotype heavy chain comprising the amino acid sequence as set forth below:

(SEQ ID NO: 82)
MQVQLVQSGAEVKKPGASVKVSCKASGFNIKDYYLHWVRQ
APGQGLEWMGWIDPDQGDTEYAQKLQGRVTMTTDTSTSTA
YMELRSLRSDDTAVYYCNAAYGSSSYPMDYWGQGTTVTVS
SASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDKTHTCPPCPAPELLG
GPSVFLFPPKPKDTLMISRTPEVTCVVVDVSHEDPEVKEN
WYVDGVEVHNAKTKPREEQYNSTYRVVSVLTVLHQDWLNG
KEYKCKVSNKALPAPIEKTISKAKGQPREPQVYTLPPSRE
EMTKNQVSLTCLVKGFYPSDIAVEWESNGQPENNYKTTPP
VLDSDGSFFLYSKLTVDKSRWQQGNVFSCSVMHEALHNHY
TQKSLSLSPGK

In a particular aspect, an anti-CD47 antibody (IgG1-5m) provided herein comprises an IgG1 heavy chain comprising the amino acid sequence as set forth below:

(SEQ ID NO: 83)
MQMQLVQSGAEVKKPGSSVKVSCKASGFNIKDYYLHWVRQ
APGQALEWMGWIDPDQGDTEYAQKFQGRVTITRDRSTSTA
YMELRSLRSEDTAVYYCNAAYGSSSYPMDYWGQGTTVTVS
SASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDKTHTCPPCPAPELLG
GPSVFLFPPKPKDTLMISRTPEVTCVVVDVSHEDPEVKEN
WYVDGVEVHNAKTKPREEQYNSTYRVVSVLTVLHQDWLNG
KEYKCKVSNKALPAPIEKTISKAKGQPREPQVYTLPPSRD
ELTKNQVSLTCLVKGFYPSDIAVEWESNGQPENNYKTTPP
VLDSDGSFFLYSKLTVDKSRWQQGNVFSCSVMHEALHNHY
TQKSLSLSPGK

In a particular aspect, an anti-CD47 antibody (IgG4P-13m) provided herein comprises an IgG4P antibody comprising the amino acid sequence as set forth below:

(SEQ ID NO: 84)
MQVQLVQSGAEVKKPGASVKVSCKASGFNIKDYYLHWVRQ
APGQGLEWMGWIDPDQGDTEYAQKLQGRVTMTTDTSTSTA
YMELRSLRSDDTAVYYCNAAYGSSSYPMDYWGQGTTVTVS
SASTKGPSVFPLAPCSRSTSESTAALGCLVKDYFPEPVTV
SWNSGALTSGVHTFPAVLQSSGLYSLSSVVTVPSSSLGTK
TYTCNVDHKPSNTKVDKRVESKYGPPCPPCPAPEFLGGPS
VFLFPPKPKDTLMISRTPEVTCVVVDVSQEDPEVQFNWYV
DGVEVHNAKTKPREEQFNSTYRVVSVLTVLHQDWLNGKEY
KCKVSNKGLPSSIEKTISKAKGQPREPQVYTLPPSQEEMT
KNQVSLTCLVKGFYPSDIAVEWESNGQPENNYKTTPPVLD
SDGSFFLYSRLTVDKSRWQEGNVFSCSVMHEALHNHYTQK
SLSLSLGK

In a particular aspect, an anti-CD47 antibody (IgG4P-5m) provided herein comprises an IgG4P heavy chain comprising the amino acid sequence as set forth below:

(SEQ ID NO: 85)
MQMQLVQSGAEVKKPGSSVKVSCKASGFNIKDYYLHWVRQ
APGQALEWMGWIDPDQGDTEYAQKFQGRVTITRDRSTSTA
YMELRSLRSEDTAVYYCNAAYGSSSYPMDYWGQGTTVTVS
SASTKGPSVFPLAPCSRSTSESTAALGCLVKDYFPEPVTV
SWNSGALTSGVHTFPAVLQSSGLYSLSSVVTVPSSSLGTK
TYTCNVDHKPSNTKVDKRVESKYGPPCPPCPAPEFLGGPS
VFLFPPKPKDTLMISRTPEVTCVVVDVSQEDPEVQFNWYV
DGVEVHNAKTKPREEQFNSTYRVVSVLTVLHQDWLNGKEY
KCKVSNKGLPSSIEKTISKAKGQPREPQVYTLPPSQEEMT
KNQVSLTCLVKGFYPSDIAVEWESNGQPENNYKTTPPVLD
SDGSFFLYSRLTVDKSRWQEGNVFSCSVMHEALHNHYTQK
SLSLSLGK

In a particular aspect, an anti-CD47 antibody (IgG4PE-13m) provided herein comprises an IgG4PE heavy chain comprising the amino acid sequence as set forth below:

(SEQ ID NO: 86)
MQVQLVQSGAEVKKPGASVKVSCKASGFNIKDYYLHWVRQ
APGQGLEWMGWIDPDQGDTEYAQKLQGRVTMTTDTSTSTA
YMELRSLRSDDTAVYYCNAAYGSSSYPMDYWGQGTTVTVS
SASTKGPSVFPLAPCSRSTSESTAALGCLVKDYFPEPVTV
SWNSGALTSGVHTFPAVLQSSGLYSLSSVVTVPSSSLGTK
TYTCNVDHKPSNTKVDKRVESKYGPPCPPCPAPEFEGGPS
VFLFPPKPKDTLMISRTPEVTCVVVDVSQEDPEVQFNWYV
DGVEVHNAKTKPREEQFNSTYRVVSVLIVLHQDWLNGKEY
KCKVSNKGLPSSIEKTISKAKGQPREPQVYTLPPSQEEMT
KNQVSLTCLVKGFYPSDIAVEWESNGQPENNYKTTPPVLD
SDGSFFLYSRLTVDKSRWQEGNVFSCSVMHEALHNHYTQK
SLSLSLGK

In a particular aspect, an anti-CD47 antibody (IgG4PE-5m) provided herein comprises an IgG4PE heavy chain comprising the amino acid sequence as set forth below:

(SEQ ID NO: 87)
MQMQLVQSGAEVKKPGSSVKVSCKASGFNIKDYYLHWVRQ
APGQALEWMGWIDPDQGDTEYAQKFQGRVTITRDRSTSTA
YMELRSLRSEDTAVYYCNAAYGSSSYPMDYWGQGTTVTVS
SASTKGPSVFPLAPCSRSTSESTAALGCLVKDYFPEPVTV
SWNSGALTSGVHTFPAVLQSSGLYSLSSVVTVPSSSLGTK
TYTCNVDHKPSNTKVDKRVESKYGPPCPPCPAPEFEGGPS
VFLFPPKPKDTLMISRTPEVTCVVVDVSQEDPEVQFNWYV
DGVEVHNAKTKPREEQFNSTYRVVSVLIVLHQDWLNGKEY
KCKVSNKGLPSSIEKTISKAKGQPREPQVYTLPPSQEEMT
KNQVSLTCLVKGFYPSDIAVEWESNGQPENNYKTTPPVLD
SDGSFFLYSRLTVDKSRWQEGNVFSCSVMHEALHNHYTQK
SLSLSLGK

In a specific aspect, provided herein is an antibody, e.g. a monoclonal antibody, which specifically binds to human CD47, wherein such an anti-CD47 antibody is a variant of a parental anti-CD47 antibody, wherein the anti-CD47 antibody, when produced using a cell-free (CF) expression system, has a higher antibody expression titer or yield compared to that of the parental anti-CD47 antibody when expressed in the CF system, and wherein the anti-CD47 antibody comprises a light chain comprising a kappa or lambda light chain constant region (e.g., human kappa or lambda light chain constant region), for example SEQ ID NO: 88.

In a specific aspect, provided herein is an antibody, e.g. a monoclonal antibody, which specifically binds to human CD47, wherein such an anti-CD47 antibody is a variant of a parental anti-CD47 antibody, wherein the anti-CD47 antibody, when produced using a cell-free (CF) expression system, has a higher antibody expression titer or yield compared to that of the parental anti-CD47 antibody when expressed in the CF system, and wherein the anti-CD47 antibody comprises (i) a VH described herein (e.g., SEQ ID NO: 89, 90, or 91) or a heavy chain described herein (e.g., any one of SEQ ID NOs:81-87), and (ii) a light chain comprising a kappa or lambda light chain constant region (e.g., human kappa or lambda light chain constant region), for example SEQ ID NO: 88, e.g., as set forth below (anti-CD47 antibody light chain (Ig K)), or SEQ ID NO: 88 without the amino acid M at the N-terminus:

(SEQ ID NO: 88)
MNIQMTQSPSAMSASVGDRVTITCKASQDIHRYLSWFQQK
PGKVPKHLIYRANRLVSGVPSRFSGSGSGTEFTLTISSLQ
PEDFATYYCLQYDEFPYTFGGGTKVEIKRTVAAPSVFIFP
PSDEQLKSGTASVVCLLNNFYPREAKVQWKVDNALQSGNS
QESVTEQDSKDSTYSLSSTLILSKADYEKHKVYACEVTHQ
GLSSPVTKSENRGEC.

In certain embodiments, an antibody described herein or an antigen-binding fragment thereof comprises a VL domain having at least 80%, at least 85%, at least 90%, at least 95%, at least 98%, or at least 99% sequence identity to the amino acid sequence of SEQ ID NO: 42, wherein the antibody specifically binds to CD47. In certain embodiments, an antibody described herein or an antigen-binding fragment thereof comprises a VL domain having at least 80%, at least 85%, at least 90%, at least 95%, at least 98%, or at least 99% sequence identity to the amino acid sequence of SEQ ID NO: 42, wherein the antibody specifically binds to CD47, and wherein the antibody comprises CDRs (e.g., VL CDRs 1-3) that are identical to the CDRs (e.g., VL CDRs 1-3) of SEQ ID NO: 42 (e.g., SEQ ID NO: 53, 71, and 55).

In certain embodiments, an antibody described herein or an antigen-binding fragment thereof comprises a light chain having at least 80%, at least 85%, at least 90%, at least 95%, at least 98%, or at least 99% sequence identity to the amino acid sequence of SEQ ID NO: 88, wherein the antibody specifically binds to CD47. In certain embodiments, an antibody described herein or an antigen-binding fragment thereof comprises a light domain having at least 80%, at least 85%, at least 90%, at least 95%, at least 98%, or at least 99% sequence identity to the amino acid sequence of SEQ ID NO: 88, wherein the antibody specifically binds to CD47, and wherein the antibody comprises CDRs (e.g., VL CDRs 1-3) that are identical to the CDRs (e.g., VL CDRs 1-3) of SEQ ID NO: 88 (e.g., SEQ ID NO: 53, 71, and 55).

In certain embodiments, an antibody described herein or an antigen-binding fragment thereof comprises a VH domain having at least 80%, at least 85%, at least 90%, at least 95%, at least 98%, or at least 99% sequence identity to the amino acid sequence of SEQ ID NO: 6, wherein the antibody specifically binds to CD47 and wherein the anti-CD47 antibody, when produced using a cell-free expression system, has a higher antibody expression titer or yield compared to the parental antibody when produced in the CF expression system. In certain embodiments, an antibody described herein or an antigen-binding fragment thereof comprises a VH domain having at least 80%, at least 85%, at least 90%, at least 95%, at least 98%, or at least 99% sequence identity to the amino acid sequence of SEQ ID NO: 6, wherein the antibody specifically binds to CD47, and wherein the antibody comprises CDRs (e.g., VL CDRs 1-3) that are identical to the CDRs (e.g., VL CDRs 1-3) of SEQ ID NO: 6 (e.g., SEQ ID NO: 50, 72, and 52).

In certain embodiments, an antibody described herein or an antigen-binding fragment thereof comprises a light chain having at least 80%, at least 85%, at least 90%, at least 95%, at least 98%, or at least 99% sequence identity to the amino acid sequence of SEQ ID NO: 78, wherein the antibody specifically binds to CD47 and wherein the anti-CD47 antibody, when produced using a cell-free expression system, has a higher antibody expression titer or yield compared to the parental antibody when produced in the CF expression system. In certain embodiments, an antibody described herein or an antigen-binding fragment thereof comprises a heavy domain having at least 80%, at least 85%, at least 90%, at least 95%, at least 98%, or at least 99% sequence identity to the amino acid sequence of SEQ ID NO: 78, wherein the antibody specifically binds to CD47, and wherein the antibody comprises CDRs (e.g., VL CDRs 1-3) that are identical to the CDRs (e.g., VL CDRs 1-3) of SEQ ID NO: 78 (e.g., SEQ ID NO: 53, 71, and 55).

In certain embodiments, an antibody described herein or an antigen-binding fragment thereof comprises a light chain having at least 80%, at least 85%, at least 90%, at least 95%, at least 98%, or at least 99% sequence identity to the amino acid sequence of SEQ ID NO: 79, wherein the antibody specifically binds to CD47 and wherein the anti-CD47 antibody, when produced using a cell-free expression system, has a higher antibody expression titer or yield compared to the parental antibody when produced in the CF expression system. In certain embodiments, an antibody described herein or an antigen-binding fragment thereof comprises a heavy domain having at least 80%, at least 85%, at least 90%, at least 95%, at least 98%, or at least 99% sequence identity to the amino acid sequence of SEQ ID NO: 79, wherein the antibody specifically binds to CD47, and wherein the antibody comprises CDRs (e.g., VL CDRs 1-3) that are identical to the CDRs (e.g., VL CDRs 1-3) of SEQ ID NO: 79 (e.g., SEQ ID NO: 53, 71, and 55).

In certain embodiments, an antibody described herein or an antigen-binding fragment thereof comprises a light chain having at least 80%, at least 85%, at least 90%, at least 95%, at least 98%, or at least 99% sequence identity to the amino acid sequence of SEQ ID NO: 80, wherein the antibody specifically binds to CD47 and wherein the anti-CD47 antibody, when produced using a cell-free expression system, has a higher antibody expression titer or yield compared to the parental antibody when produced in the CF expression system. In certain embodiments, an antibody described herein or an antigen-binding fragment thereof comprises a heavy domain having at least 80%, at least 85%, at least 90%, at least 95%, at least 98%, or at least 99% sequence identity to the amino acid sequence of SEQ ID NO: 80, wherein the antibody specifically binds to CD47, and wherein the antibody comprises CDRs (e.g., VL CDRs 1-3) that are identical to the CDRs (e.g., VL CDRs 1-3) of SEQ ID NO: 80 (e.g., SEQ ID NO: 53, 71, and 55).

Table 3 provides a table of the anti-CD47 antibody amino acid sequences described herein. In certain embodiments, an antibody described herein comprises any light chain variable region sequence from Table 3 and any heavy chain variable region sequence from Table 3.

TABLE 3
Anti-CD47 antibody amino acid sequences.

| SEQ | | |
|---|---|---|
| ID | | |
| NO: | DESCRIPTION | Amino Acid Sequence |
| 1 | IgG1 | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP EYKCKVSNKA LPAPIEKTIS |
| | | KAKGQPREPQ VYTLPPSRDE LTKNQVSLTC LVKGFYPSDI |
| | | AVEWESNGQP ENNYKTTPPV LDSDGSFFLY SKLTVDKSRW |
| | | QQGNVFSCSV MHEALHNHYT QKSLSLSPGK |
| 2 | IgG2 | ASTKGPSVFP LAPCSRSTSE STAALGCLVK DYFPEPVTVS |
| | | WNSGALTSGV HTFPAVLQSS GLYSLSSVVT VPSSNFGTQT |
| | | YTCNVDHKPS NTKVDKTVER KCCVECPPCP APPVAGPSVF |
| | | LFPPKPKDTL MISRTPEVTC VVVDVSHEDP KVSNKGLPAP |
| | | IEKTISKTKG QPREPQVYTL PPSREEMTKN QVSLTCLVKG |
| | | FYPSDISVEW ESNGQPENNY KTTPPMLDSD GSFFLYSKLT |
| | | VDKSRWQQGN VFSCSVMHEA LHNHYTQKSL SLSPGK |
| 3 | IgG3 | ASTKGPSVFP LAPCSRSTSG GTAALGCLVK DYFPEPVTVS |
| | | WNSGALTSGV HTFPAVLQSS GLYSLSSVVT VPSSSLGTQT |
| | | YTCNVNHKPS NTKVDKRVEL KTPLGDTTHT CPRCPEPKSC |
| | | DTPPPCPRCP EPKSCDTPPP CPRCPEPKSC DTPPPCPRCP |
| | | APELLGGPSV FLFPPKPKDT LMISRTPEVT CVVVDVSHED |
| | | CKVSNKALPA PIEKTISKTK GQPREPQVYT LPPSREEMTK |
| | | NQVSLTCLVK GFYPSDIAVE WESSGQPENN YNTTPPMLDS |
| | | DGSFFLYSKL TVDKSRWQQG |
| 4 | IgG4 | ASTKGPSVFP LAPCSRSTSE STAALGCLVK DYFPEPVTVS |
| | | WNSGALTSGV HTFPAVLQSS GLYSLSSVVT VPSSSLGTKT |
| | | YTCNVDHKPS NTKVDKRVES CKVSNKGLPS SIEKTISKAK |
| | | GQPREPQVYT LPPSQEEMTK NQVSLTCLVK GFYPSDIAVE |
| | | WESNGQPENN YKTTPPVLDS DGSFFLYSRL TVDKSRWQEG |
| | | NVFSCSVMHE ALHNHYTQKS LSLSLGK |
| 5 | Variable | EVQLQQSGAE LVRSGASVKL SCTASGENIK DYYLHWVKQR |
| | heavy | PEQGLEWIGW IDPDNGDTEF APKFQGKATM TADTSSNTAY |
| | chain | LQLSSLTSED TAVYYCNAAY GSSSYPMDYW GQGTSVTV |
| | region of | |
| | a | |
| | humanized | |
| | CD47 | |
| | antibody | |
| 6 | Anti-CD47 | QMQLVQSGAE VKKTGSSVKV SCKASGENIK DYYLHWVRQA |
| | antibody | PGQALEWMGW IDPDQGDTEY AQKFQDRVTI TRDRSMSTAY |
| | AB6.12 | MELSSLRSED TAMYYCNAAY GSSSYPMDYW GQGTTVTV |
| | heavy | |
| | chain | |
| | variable | |
| | region | |
| 7 | Variable | QMQLVQSGAE VKKTGSSVKV SCKASGENIK DYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 8 | Variable | EVQLVQSGAE VKKTGSSVKV SCKASGENIK DYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 9 | Variable | QMQLVQSGAE VKKTGSSVKV SCKASGENIK DYYLHWVRQA |
| | heavy | PGQALEWMGW IDPDNGDTEY AQKFQGRVTM TADTSSNTAY |
| | chain | MELSSLRSED TAMYYCNAAY GSSSYPMDYW GQGTTVTV |
| | region of | |
| | a | |
| | humanized | |
| | CD47 | |
| | antibody | |
| 10 | Variable | QMQLVQSGAE VKKTGSSVKV SCKASGENIK DYYLHWVRQA |
| | heavy | PGQALEWMGW IDPDNGDTEY AQKFQGRVTM TEDTSTDTAY |
| | chain | MELSSLRSED TAMYYCNAAY GSSSYPMDYW GQGTTVTV |
| | region of | |
| | a | |
| | humanized | |
| | CD47 | |
| | antibody | |
| 11 | Variable | QMQLVQSGAE VKKTGSSVKV SCKASGENIK DYYLHWVRQA |
| | heavy | PGQALEWMGW IDPDQGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 12 | Variable | QMQLVQSGAE VKKTGSSVKV SCKASGENIK DYYLHWVRQA |
| | heavy | PGQALEWMGW IDPDYGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 13 | Variable | QMQLVQSGAE VKKTGSSVKV SCKASGENIK DYYLHWVRQA |
| | heavy | PGQALEWMGW IDPDSGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 14 | Variable | QMQLVQSGAE VKKTGSSVKV SCKASGENIK DYYLHWVRQA |
| | heavy | PGQALEWMGW IDPDNADTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 15 | Variable | QMQLVQSGAE VKKTGSSVKV SCKASGENIK DYYLHWVRQA |
| | heavy | PGQALEWMGW IDPDNTDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 16 | Variable | QMQLVQSGAE VKKTGSSVKV SCKASGFNIK DYYLHWVRQA |
| | heavy | PGQALEWMGW IDPDNGDTEY AQKFQDRVTI TRDRSMSTAY |
| | chain | MELSSLRSED TAMYYCNAAY GSSPYPMDYW GQGTTVTV* |
| | region of | |
| | a | |
| | humanized | |
| | CD47 | |
| | antibody | |
| 17 | Variable | QMQLVQSGAE VKKTGSSVKV SCKASGYTFT YYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 18 | Variable | QMQLVQSGAE VKKTGSSVKV SCKASGFTFT YYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 19 | Variable | QMQLVQSGAE VKKTGSSVKV SCKASGYNFT YYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 20 | Variable | QMQLVQSGAE VKKTGSSVKV SCKASGYTIT YYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 21 | Variable | QMQLVQSGAE VKKTGSSVKV SCKASGYTFK YYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 22 | Variable | QMQLVQSGAE VKKTGSSVKV SCKASGYTFT DYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 23 | Variable | QMQLVQSGAE VKKTGSSVKV SCKASGFTFT DYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 24 | Variable | QMQLVQSGAE VKKTGSSVKV SCKASGFTIT DYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 25 | Variable | QMQLVQSGAE VKKTGSSVKV SCKASGYTFK DYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 26 | Variable | QMQLVQSGAE VKKTGSSVKV SCKASGFTFK DYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 27 | Variable | QMQLVQSGAE VKKTGSSVKV SCKASGENIK DYYLHWVRQA |
| | heavy | PGQALEWMGW IDPDNGDTEY AQKFQDRVTI TRDRSMSTAY |
| | chain | LQLSSLRSED TAMYYCNAAY GSSSYPMDYW GQGTTVTV |
| | region of | |
| | a | |
| | humanized | |
| | CD47 | |
| | antibody | |
| 28 | Variable | QMQLVQSGAE VKKTGSSVKV SCKASGENIK DYYLHWVRQA |
| | heavy | PGQALEWMGW IDPDNGDTEY AQKFQDRVTI TRDRSMSTAY |
| | chain | MELSSLTSED TAVYYCNAAY GSSSYPMDYW GQGTTVTV |
| | region of | |
| | a | |
| | humanized | |
| | CD47 | |
| | antibody | |
| 29 | Variable | EVQLVQSGAE VKKPGATVKI SCKVSGFNIK DYYLHWVRQA |
| | heavy | PGQALEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 30 | Variable | EVQLVQSGAE VKKPGATVKI SCKVSGENIK DYYLHWVQQA |
| | heavy | PGKGLEWMGW IDPDNGDTEY AQKFQDRVTI TRDRSMSTAY |
| | chain | MELSSLRSED TAMYYCNAAY GSSSYPMDYW GQGTTVTV |
| | region of | |
| | a | |
| | humanized | |
| | CD47 | |
| | antibody | |
| 31 | Variable | DIKMTQSPSS LYASLGERVT ITCKASQDIH RYLSWFQQKP |
| | light | GKSPKILIYR ANRLVDGVPS RFSGSGSGQD YSLTISSLEY |
| | chain | EDMGIYYCLQ YDEFPYTFGG GTKLEMK |
| | region of | |
| | a | |
| | humanized | |
| | CD47 | |
| | antibody | |
| 32 | Variable | DIKMTQSPSS LYASLGERVT ITCKASQDIH RYLSWFQQKP |
| | light | GKSPKILIYR ANRLVDGVPS RFSGSGSGQD YSLTISSLEY |
| | chain | EDMGIYYCLQ YDEFPYTFGG GTKLEIK |
| | region of | |
| | a | |
| | humanized | |
| | CD47 | |
| | antibody | |
| 33 | Variable | DIQMTQSPSS LSASVGDRVT ITCKASQDIH RYLSWYQQKP |
| | light | GKAPKLLIYR ANRLVDGVPS RFSGSGSGTD FTFTISSLQP |
| | chain | EDIATYYCLQ YDEFPYTFGG GTKVEIK |
| | region of | |
| | a | |
| | humanized | |
| | CD47 | |
| | antibody | |
| 34 | Variable | DIQMTQSPSS LSASVGDRVT ITCKASQDIH RYLSWFQQKP |
| | light | GKAPKSLIYR ANRLVDGVPS RFSGSGSGTD FTLTISSLQP |
| | chain | EDFATYYCLQ YDEFPYTEGG GTKVEIK |
| | region of | |
| | a | |
| | humanized | |
| | CD47 | |
| | antibody | |
| 35 | Variable | NIQMTQSPSA MSASVGDRVT ITCKASQDIH RYLSWFQQKP |
| | light | GKVPKHLIYR ANRLVDGVPS RFSGSGSGTE FTLTISSLQP |
| | chain | EDFATYYCLQ YDEFPYTEGG GTKVEIK |
| | region of | |
| | a | |
| | humanized | |
| | CD47 | |
| | antibody | |
| 36 | Variable | DIQMTQSPSS LSASVGDRVT ITCKASQDIH RYLSWYQQKP |
| | light | GKAPKRLIYR ANRLVDGVPS RFSGSGSGTE FTLTISSLQP |
| | chain | EDFATYYCLQ YDEFPYTFGG GTKVEIK |
| | region of | |
| | a | |
| | humanized | |
| | CD47 | |
| | antibody | |
| 37 | Variable | DIQMTQSPSS LSASVGDRVT ITCRASQDIH RYLAWYQQKP |
| | light | GKVPKLLIYR ANRLQSGVPS RFSGSGSGTD FTLTISSLQP |
| | chain | EDVATYYCLQ YDEFPYTFGQ GTKVEIK |
| | region of | |
| | a | |
| | humanized | |
| | CD47 | |
| | antibody | |
| 38 | Variable | EIVLTQSPAT LSLSPGERAT LSCRASQDIH RYLAWYQQKP |
| | light | GQAPRLLIYR ANRRATGIPA RFSGSGSGTD FTLTISSLEP |
| | chain | EDFAVYYCLQ YDEFPYTGFQ GTRLEIK |
| | region of | |
| | a | |
| | humanized | |
| | CD47 | |
| | antibody | |
| 39 | Variable | DIQMTQSPSA MSASVGDRVT ITCKASQDIH RYLSWFQQKP |
| | light | GKVPKHLIYR ANRLVDGVPS RFSGSGSGTE FTLTISSLQP |
| | chain | EDFATYYCLQ YDEFPYTEGG GTKVEIK |
| | region of | |
| | a | |
| | humanized | |
| | CD47 | |
| | antibody | |
| 40 | Variable | NIQMTQSPSA MSASVGDRVT ITCRARQGIH RYLSWFQQKP |
| | light | GKVPKHLIYR ANRLVDGVPS RFSGSGSGTE FTLTISSLQP |
| | chain | EDFATYYCLQ YDEFPYTEGG GTKVEIK |
| | region of | |
| | a | |
| | humanized | |
| | CD47 | |
| | antibody | |
| 41 | Variable | NIQMTQSPSA MSASVGDRVT ITCKASQDIH RYLSWFQQKP |
| | light | GKVPKILIYR ANRLVDGVPS RFSGSGSGTE FTLTISSLQP |
| | chain | EDFATYYCLQ YDEFPYTFGG GTKVEIK |
| | region of | |
| | a | |
| | humanized | |
| | CD47 | |
| | antibody | |
| 42 | Anti-CD47 | NIQMTQSPSA MSASVGDRVT ITCKASQDIH RYLSWFQQKP |
| | antibody | GKVPKHLIYR ANRLVSGVPS RESGSGSGTE FTLTISSLQP |
| | AB6.12 | EDFATYYCLQ YDEFPYTEGG GTKVEIK |
| | light | |
| | chain | |
| | variable | |
| | region | |
| 43 | Variable | NIQMTQSPSA MSASVGDRVT ITCRARQGIH RYLSWFQQKP |
| | light | GKVPKILIYR ANRLVDGVPS RFSGSGSGTE FTLTISSLQP |
| | chain | EDFATYYCLQ YDEFPYTFGG GTKVEIK |
| | region of | |
| | a | |
| | humanized | |
| | CD47 | |
| | antibody | |
| 44 | Variable | NIQMTQSPSA MSASVGDRVT ITCRARQGIH RYLSWFQQKP |
| | light | GKVPKHLIYR ANRLVSGVPS RFSGSGSGTE FTLTISSLQP |
| | chain | EDFATYYCLQ YDEFPYTFGG GTKVEIK |
| | region of | |
| | a | |
| | humanized | |
| | CD47 | |
| | antibody | |
| 45 | Variable | NIQMTQSPSA MSASVGDRVT ITCKASQDIH RYLSWFQQKP |
| | light | GKVPKLLIYR ANRLVDGVPS RFSGSGSGTE FTLTISSLQP |
| | chain | EDFATYYCLQ YDEFPYTFGG GTKVEIK |
| | region of | |
| | a | |
| | humanized | |
| | CD47 | |
| | antibody | |
| 46 | Variable | NIQMTQSPSA MSASVGDRVT ITCKASQDIH RYLSWFQQKP |
| | light | GKVPKLLIYR ANRLVSGVPS RFSGSGSGTE FTLTISSLQP |
| | chain | EDFATYYCLQ YDEFPYTEGG GTKVEIK |
| | region of | |
| | a | |
| | humanized | |
| | CD47 | |
| | antibody | |
| 47 | Variable | NIQMTQSPSA MSASVGDRVT |
| | light | ITCRARQGIH RYLSWFQQKP |
| | chain | GKVPKLLIYR ANRLVSGVPS |
| | region of | RFSGSGSGTE FTLTISSLQP |
| | a | EDFATYYCLQ YDEFPYTFGG |
| | humanized | GTKVEIK |
| | CD47 | |
| | antibody | |
| 48 | EXEMPLARY | MWPLVAALLL GSACCGSAQL LENKTKSVEF TFCNDTVVIP |
| | HUMAN CD47 | CFVTNMEAQN TTEVYVKWKF KGRDIYTEDG ALNKSTVPTD |
| | SEQUENCE | FSSAKIEVSQ LLKGDASLKM DKSDAVSHTG NYTCEVTELT |
| | | REGETIIELK YRVVSWFSPN ENILIVIFPI FAILLFWGQF |
| | | GIKTLKYRSG GMDEKTIALL VAGLVITVIV IVGAILFVPG |
| | | EYSLKNATGL GLIVISTGIL ILLHYYVEST AIGLTSFVIA |
| | | ILVIQVIAYI LAVVGLSLCI AACIPMHGPL LISGLSILAL |
| | | AQLLGLVYMK FVASNQKTIQ PPRKAVEEPL NAFKESKGMM |
| | | NDE |
| 49 | EXEMPLARY | QLLFNKTKSV EFTFCNDTVV IPCFVTNMEA QNTTEVYVKW |
| | HUMAN CD47 | KFKGRDIYTF DGALNKSTVP TDESSAKIEV SQLLKGDASL |
| | SEQUENCE | KMDKSDAVSH TGNYTCEVTE LIREGETIIE LKYRVVSWFS |
| | WITHOUT | PNENILIVIF PIFAILLFWG QFGIKTLKYR SGGMDEKTIA |
| | SIGNAL | LLVAGLVITV IVIVGAILFV PGEYSLKNAT GLGLIVISTG |
| | SEQUENCE | ILILLHYYVF STAIGLISFV IAILVIQVIA YILAVVGLSL |
| | | CIAACIPMHG PLLISGLSIL ALAQLLGLVY MKFVASNQKT |
| | | IQPPRKAVEE PLNAFKESKG MMNDE |
| 50 | amino acid | GFNIKDYYLH |
| | sequence | |
| | of VH CDR1 | |
| 51 | amino acid | WIDPDNGDTE |
| | sequence | |
| | of VH CDR2 | |
| 52 | amino acid | NAAYGSSSYPMDY |
| | sequence | |
| | of VH CDR3 | |
| 53 | amino acid | KASQDIHRYLS |
| | sequence | |
| | of VL CDR1 | |
| 54 | amino acid | RANRLVD |
| | sequence | |
| | of VL CDR2 | |
| 55 | amino acid | LQYDEFPYT |
| | sequence | |
| | of VL CDR3 | |
| 56 | CD47 | KGRD |
| | epitope | |
| 57 | amino acid | GYTFTYYYLH |
| | sequence | |
| | of VH CDR1 | |
| 58 | amino acid | GFTFTYYYLA |
| | sequence | |
| | of VH CDR1 | |
| 59 | amino acid | GYNFTYYYLH |
| | sequence | |
| | of VH CDR1 | |
| 60 | amino acid | GYTITYYYLH |
| | sequence | |
| | of VH CDR1 | |
| 61 | amino acid | GYTFKYYYLH |
| | sequence | |
| | of VH CDR1. | |
| 62 | amino acid | GYTFTDYYLH |
| | sequence | |
| | of VH CDR1 | |
| 63 | amino acid | GFTFTDYYLH |
| | sequence | |
| | of VH CDR1 | |
| 64 | amino acid | GFTITDYYLA |
| | sequence | |
| | of VH CDR1 | |
| 65 | amino acid | GYTFKDYYLH |
| | sequence | |
| | of VH CDR1 | |
| 66 | amino acid | GFTFKDYYLH |
| | sequence | |
| | of VH CDR1 | |
| 67 | amino acid | RASQDIHRYLA |
| | sequence | |
| | of VL CDR1 | |
| 68 | amino acid | RARQGIHRYLS |
| | sequence | |
| | of VL CDR1 | |
| 69 | amino acid | RANRIQS |
| | sequence | |
| | Of VI CDR2 | |
| 70 | amino acid | RANRRAT |
| | sequence | |
| | of VL CDR2 | |
| 71 | amino acid | RANRLVS |
| | sequence | |
| | of VL CDR2 | |
| 72 | amino acid | WIDPDQGDTE |
| | sequence | |
| | of VH CDR2 | |
| 73 | amino acid | WIDPDYGDTE |
| | sequence | |
| | of VH CDR2 | |
| 74 | amino acid | WIDPDSGDTE |
| | sequence | |
| | of VH CDR2 | |
| 75 | amino acid | WIDPDNADTE |
| | sequence | |
| | of VH CDR2 | |
| 76 | amino acid | WIDPDNIDTE |
| | sequence | |
| | of VH CDR2 | |
| 77 | amino acid | NAAYGSSPYPMDY |
| | sequence | |
| | of VH CDR3 | |
| 78 | Anti-CD47 | MQMQLVQSGA EVKKTGSSVK VSCKASGENI KDYYLHWVRQ |
| | antibody | APGQALEWMG WIDPDQGDTE YAQKFQDRVT ITRDRSMSTA |
| | IgG1 heavy | YMELSSLRSE DTAMYYCNAA YGSSSYPMDY WGQGTTVTVS |
| | chain | SASTKGPSVF PLAPSSKSTS GGTAALGCLV KDYFPEPVTV |
| | | SWNSGALTSG VHTFPAVLQS SGLYSLSSVV TVPSSSLGTQ |
| | | TYICNVNHKP SNTKVDKKVE PKSCDKTHTC PPCPAPELLG |
| | | GPSVFLFPPK PKDTLMISRT PEVTCVVVDV SHEDPEVKEN |
| | | WYVDGVEVHN AKIKPREEQY NSTYRVVSVL TVLHQDWLNG |
| | | KEYKCKVSNK ALPAPIEKTI SKAKGQPREP QVYTLPPSRD |
| | | ELTKNQVSLT CLVKGFYPSD IAVEWESNGQ PENNYKITPP |
| | | VLDSDGSFFL YSKLTVDKSR WQQGNVFSCS VMHEALHNHY |
| | | TQKSLSLSPG K |
| 79 | Anti-CD47 | MQMQLVQSGA EVKKTGSSVK VSCKASGENI KDYYLHWVRQ |
| | antibody | APGQALEWMG WIDPDQGDTE YAQKFQDRVT ITRDRSMSTA |
| | IgG4P | YMELSSLRSE DTAMYYCNAA YGSSSYPMDY WGQGTTVTVS |
| | heavy | SASTKGPSVF PLAPCSRSTS ESTAALGCLV KDYFPEPVTV |
| | chain | SWNSGALTSG VHTFPAVLQS SGLYSLSSVV TVPSSSLGTK |
| | | TYTCNVDHKP SNTKVDKRVE SKYGPPCPPC PAPEFLGGPS |
| | | VFLFPPKPKD TLMISRTPEV TCVVVDVSQE DPEVQFNWYV |
| | | DGVEVHNAKT KPREEQFNST YRVVSVLTVL HQDWLNGKEY |
| | | KCKVSNKGLP SSIEKTISKA KGQPREPQVY TLPPSQEEMT |
| | | KNQVSLTCLV KGFYPSDIAV EWESNGQPEN NYKTTPPVLD |
| | | SDGSFFLYSR LTVDKSRWQE GNVFSCSVMH EALHNHYTQK |
| | | SLSLSLGK |
| 80 | Anti-CD47 | MQMQLVQSGA EVKKTGSSVK VSCKASGENI KDYYLHWVRQ |
| | antibody | APGQALEWMG WIDPDQGDTE YAQKFQDRVT ITRDRSMSTA |
| | IgG4PE | YMELSSLRSE DTAMYYCNAA YGSSSYPMDY WGQGTTVTVS |
| | heavy | SASTKGPSVF PLAPCSRSTS ESTAALGCLV KDYFPEPVTV |
| | chain | SWNSGALTSG VHTFPAVLQS SGLYSLSSVV TVPSSSLGTK |
| | (comprisin | TYTCNVDHKP SNTKVDKRVE SKYGPPCPPC PAPEFEGGPS |
| | g S228P | VFLFPPKPKD TLMISRTPEV TCVVVDVSQE DPEVQFNWYV |
| | and L235E | DGVEVHNAKT KPREEQFNST YRVVSVLIVL HQDWLNGKEY |
| | substituti | KCKVSNKGLP SSIEKTISKA KGQPREPQVY TLPPSQEEMT |
| | ons) | KNQVSLTCLV KGFYPSDIAV EWESNGQPEN NYKTTPPVLD |
| | | SDGSFFLYSR LTVDKSRWQE GNVFSCSVMH EALHNHYTQK |
| | | SLSLSLGK |
| 81 | Anti-CD47 | MQVQLVQSGA EVKKPGASVK VSCKASGENI KDYYLHWVRQ |
| | antibody | APGQGLEWMG WIDPDQGDTE YAQKLQGRVT MTTDISTSTA |
| | IgG1-13m | YMELRSLRSD DTAVYYCNAA YGSSSYPMDY WGQGTTVTVS |
| | heavy | SASTKGPSVF PLAPSSKSTS GGTAALGCLV KDYFPEPVTV |
| | chain | SWNSGALTSG VHTFPAVLQS SGLYSLSSVV TVPSSSLGTQ |
| | | TYICNVNHKP SNTKVDKKVE PKSCDKTHTC PPCPAPELLG |
| | | GPSVFLFPPK PKDTLMISRT PEVTCVVVDV SHEDPEVKEN |
| | | WYVDGVEVHN AKTKPREEQY NSTYRVVSVL TVLHQDWLNG |
| | | KEYKCKVSNK ALPAPIEKTI SKAKGQPREP QVYTLPPSRD |
| | | ELTKNQVSLT CLVKGFYPSD IAVEWESNGQ PENNYKTTPP |
| | | VLDSDGSFFL YSKLTVDKSR WQQGNVESCS VMHEALHNHY |
| | | TQKSLSLSPG K |
| 82 | Anti-CD47 | MQVQLVQSGA EVKKPGASVK VSCKASGENI KDYYLHWVRQ |
| | antibody | APGQGLEWMG WIDPDQGDTE YAQKLQGRVT MTTDTSTSTA |
| | IgG1-13mZ | YMELRSLRSD DTAVYYCNAA YGSSSYPMDY WGQGTTVTVS |
| | heavy | SASTKGPSVF PLAPSSKSTS GGTAALGCLV KDYFPEPVTV |
| | chain | SWNSGALTSG VHTFPAVLQS SGLYSLSSVV TVPSSSLGTQ |
| | | TYICNVNHKP SNTKVDKKVE PKSCDKTHTC PPCPAPELLG |
| | | GPSVFLFPPK PKDTLMISRT PEVTCVVVDV SHEDPEVKEN |
| | | WYVDGVEVHN AKTKPREEQY NSTYRVVSVL TVLHQDWLNG |
| | | KEYKCKVSNK ALPAPIEKTI SKAKGQPREP QVYTLPPSRE |
| | | EMTKNQVSLT CLVKGFYPSD IAVEWESNGQ PENNYKTTPP |
| | | VLDSDGSFFL YSKLTVDKSR WQQGNVESCS VMHEALHNHY |
| | | TQKSLSLSPGK |
| 83 | Anti-CD47 | MQMQLVQSGA EVKKPGSSVK VSCKASGENI KDYYLHWVRQ |
| | antibody | APGQALEWMG WIDPDQGDTE YAQKFQGRVT ITRDRSTSTA |
| | IgG1-5m | YMELRSLRSE DTAVYYCNAA YGSSSYPMDY WGQGTTVTVS |
| | heavy | SASTKGPSVF PLAPSSKSTS GGTAALGCLV KDYFPEPVTV |
| | chain | SWNSGALTSG VHTFPAVLQS SGLYSLSSVV TVPSSSLGTQ |
| | | TYICNVNHKP SNTKVDKKVE PKSCDKTHTC PPCPAPELLG |
| | | GPSVFLFPPK PKDTLMISRT PEVTCVVVDV SHEDPEVKEN |
| | | WYVDGVEVHN AKTKPREEQY NSTYRVVSVL TVLHQDWLNG |
| | | KEYKCKVSNK ALPAPIEKTI SKAKGQPREP QVYTLPPSRD |
| | | ELTKNQVSLT CLVKGFYPSD IAVEWESNGQ PENNYKTTPP |
| | | VLDSDGSFFL YSKLTVDKSR WQQGNVESCS VMHEALHNHY |
| | | TQKSLSLSPG K |
| 84 | Anti-CD47 | MQVQLVQSGA EVKKPGASVK VSCKASGFNI KDYYLHWVRQ |
| | antibody | APGQGLEWMG WIDPDQGDTE YAQKLQGRVT MITDISTSTA |
| | IgG4P-13m | YMELRSLRSD DTAVYYCNAA YGSSSYPMDY WGQGTTVTVS |
| | heavy | SASTKGPSVF PLAPCSRSTS ESTAALGCLV KDYFPEPVTV |
| | chain | SWNSGALTSG VHTFPAVLQS SGLYSLSSVV TVPSSSLGTK |
| | | TYTCNVDHKP SNTKVDKRVE SKYGPPCPPC PAPEFLGGPS |
| | | VFLFPPKPKD TLMISRTPEV TCVVVDVSQE DPEVQFNWYV |
| | | DGVEVHNAKT KPREEQFNST YRVVSVLTVL HQDWLNGKEY |
| | | KCKVSNKGLP SSIEKTISKA KGQPREPQVY TLPPSQEEMT |
| | | KNQVSLTCLV KGFYPSDIAV EWESNGQPEN NYKTTPPVLD |
| | | SDGSFFLYSR LTVDKSRWQE GNVFSCSVMH EALHNHYTQK |
| | | SLSLSLGK |
| 85 | Anti-CD47 | MQMQLVQSGA EVKKPGSSVK VSCKASGENI KDYYLHWVRQ |
| | antibody | APGQALEWMG WIDPDQGDTE YAQKFQGRVT ITRDRSTSTA |
| | IgG4P-5m | YMELRSLRSE DTAVYYCNAA YGSSSYPMDY WGQGTTVTVS |
| | heavy | SASTKGPSVF PLAPCSRSTS ESTAALGCLV KDYFPEPVTV |
| | chain | SWNSGALTSG VHTFPAVLQS SGLYSLSSVV TVPSSSLGTK |
| | | TYTCNVDHKP SNTKVDKRVE SKYGPPCPPC PAPEFLGGPS |
| | | VFLFPPKPKD TLMISRTPEV TCVVVDVSQE DPEVQFNWYV |
| | | DGVEVHNAKT KPREEQFNST YRVVSVLTVL HQDWLNGKEY |
| | | KCKVSNKGLP SSIEKTISKA KGQPREPQVY TLPPSQEEMT |
| | | KNQVSLTCLV KGFYPSDIAV EWESNGQPEN NYKTTPPVLD |
| | | SDGSFFLYSR LTVDKSRWQE GNVFSCSVMH EALHNHYTQK |
| | | SLSLSLGK |
| 86 | Anti-CD47 | MQVQLVQSGA EVKKPGASVK VSCKASGENI KDYYLHWVRQ |
| | antibody | APGQGLEWMG WIDPDQGDTE YAQKLQGRVT MTTDTSTSTA |
| | IgG4PE-13m | YMELRSLRSD DTAVYYCNAA YGSSSYPMDY WGQGTTVTVS |
| | heavy | SASTKGPSVF PLAPCSRSTS ESTAALGCLV KDYFPEPVTV |
| | chain | SWNSGALTSG VHTFPAVLQS SGLYSLSSVV TVPSSSLGTK |
| | | TYTCNVDHKP SNTKVDKRVE SKYGPPCPPC PAPEFEGGPS |
| | | VFLFPPKPKD TLMISRTPEV TCVVVDVSQE DPEVQFNWYV |
| | | DGVEVHNAKT KPREEQFNST YRVVSVLTVL HQDWLNGKEY |
| | | KCKVSNKGLP SSIEKTISKA KGQPREPQVY TLPPSQEEMT |
| | | KNQVSLTCLV KGFYPSDIAV EWESNGQPEN NYKTTPPVLD |
| | | SDGSFFLYSR LTVDKSRWQE GNVFSCSVMH EALHNHYTQK |
| | | SLSLSLGK |
| 87 | Anti-CD47 | MQMQLVQSGA EVKKPGSSVK VSCKASGENI KDYYLHWVRQ |
| | antibody | APGQALEWMG WIDPDQGDTE YAQKFQGRVT ITRDRSTSTA |
| | IgG4PE-5m | YMELRSLRSE DTAVYYCNAA YGSSSYPMDY WGQGTTVTVS |
| | heavy | SASTKGPSVF PLAPCSRSTS ESTAALGCLV KDYFPEPVTV |
| | chain | SWNSGALTSG VHTFPAVLQS SGLYSLSSVV TVPSSSLGTK |
| | | TYTCNVDHKP SNTKVDKRVE SKYGPPCPPC PAPEFEGGPS |
| | | VFLFPPKPKD TLMISRTPEV TCVVVDVSQE DPEVQFNWYV |
| | | DGVEVHNAKT KPREEQFNST YRVVSVLTVL HQDWLNGKEY |
| | | KCKVSNKGLP SSIEKTISKA KGQPREPQVY TLPPSQEEMT |
| | | KNQVSLTCLV KGFYPSDIAV EWESNGQPEN NYKTTPPVLD |
| | | SDGSFFLYSR LTVDKSRWQE GNVFSCSVMH EALHNHYTQK |
| | | SLSLSLGK |
| 88 | Light | MNIQMTQSPS AMSASVGDRV TITCKASQDI HRYLSWFQQK |
| | chain (Ig | PGKVPKHLIY RANRLVSGVP SRFSGSGSGT EFTLTISSLQ |
| | Kappa) | PEDFATYYCL QYDEFPYTFG GGTKVEIKRT VAAPSVFIFP |
| | | PSDEQLKSGT ASVVCLLNNF YPREAKVQWK VDNALQSGNS |
| | | QESVTEQDSK DSTYSLSSTL TLSKADYEKH KVYACEVTHQ |
| | | GLSSPVTKSF NRGEC |
| 89 | consensus | X₁QX₂QLVQSGAEVKKX₃GX₄SVKVSCKASGFNIKDYYLHWVRQA |
| | sequence | PGQX₅LEWMGWIDPDQGDTEYAQKX₆QX₇RVTX₈TX₉DX₁₀SX₁₁S |
| | for anti- | TAYMELX₁₂SLRSX₁₃DTAX₁₄YYCNAAYGSSSYPMDYWGQGTTV |
| | CD47 | TV |
| | antibody | |
| | heavy | |
| | chain | |
| | variable | |
| | region | |
| 90 | anti-CD47 | MQVQLVQSGA EVKKPGASVK VSCKASGENI KDYYLHWVRQ |
| | antibody | APGQGLEWMG WIDPDQGDTE YAQKLQGRVT MTTDTSTSTA |
| | VH-13m | YMELRSLRSD DTAVYYCNAA YGSSSYPMDY WGQGTTVTV |
| 91 | anti-CD47 | MQMQLVQSGA EVKKPGSSVK VSCKASGENI KDYYLHWVRQ |
| | antibody | APGQALEWMG WIDPDQGDTE YAQKLQGRVT MTTDTSTSTA |
| | VH-5m | YMELRSLRSD DTAVYYCNAA YGSSSYPMDY WGQGTTVTV |

In some embodiments, the CD47 antibodies described herein are used in conjunction with one or more additional agents or a combination of additional agents. Suitable additional agents include current pharmaceutical and/or surgical therapies for an intended application, such as, for example, cancer. For example, the CD47 antibodies can be used in conjunction with one or more additional chemotherapeutic or anti-neoplastic agents. Alternatively, the additional chemotherapeutic agent is radiotherapy. In some embodiments, the chemotherapeutic agent is a cell death-inducing agent. In some embodiments, the chemotherapeutic agent induces a loss of phospholipid asymmetry across the plasma membrane, for example causes cell surface exposure of phosphatidylserine (PS). In some embodiments, the chemotherapeutic agent induces endoplasmic reticulum (ER) stress. In some embodiments, the chemotherapeutic agent is a proteasome inhibitor. In some embodiments, the chemotherapeutic agent induces the translocation of ER proteins to the cell surface. In some embodiments, the chemotherapeutic agent induces the translocation and cell surface exposure of calreticulin.

In some embodiments, the CD47 antibody and additional agent are formulated into a single therapeutic composition, and the CD47 antibody and additional agent are administered simultaneously. Alternatively, the CD47 antibody and additional agent are separate from each other, e.g., each is formulated into a separate therapeutic composition, and the CD47 antibody and the additional agent are administered simultaneously, or the CD47 antibody and the additional agent are administered at different times during a treatment regimen. For example, the CD47 antibody is administered prior to the administration of the additional agent, the CD47 antibody is administered subsequent to the administration of the additional agent, or the CD47 antibody and the additional agent are administered in an alternating fashion. As described herein, the CD47 antibody and additional agent are administered in single doses or in multiple doses.

In particular aspects, anti-CD47 antibodies provided herein comprise one or more non-natural amino acid residues at site-specific positions. See, e.g., U.S. Application Publication No. US 2014/0046030 A1, which is incorporated herein by reference in its entirety. In specific aspects, non-natural amino acid residues at site specific positions has advantages for antibody production yield, solubility, binding affinity, and/or activity. Non-limiting examples of non-natural amino acids have been described, see, e.g., U.S. Application Publication No. US 2014/0066598 A1.

In a particular aspect, provided herein are anti-CD47 antibodies conjugated to a conjugation moiety or an agent such as a label or toxin. A conjugation moiety can be any conjugation moiety deemed useful to one of skill in the art. For instance, a conjugation moiety can be a polymer, such as polyethylene glycol, that can improve the stability of the antibody in vitro or in vivo. A conjugation moiety can have therapeutic activity, thereby yielding an antibody-drug conjugate. A conjugation moiety can be a molecular payload that is harmful to target cells. A conjugation moiety can be a label useful for detection or diagnosis. In certain aspects, a conjugation moiety is linked to the antibody via a direct covalent bond. In certain aspects, a conjugation moiety is linked to the antibody via a linker. In particular aspects, a conjugation moiety or a linker is attached via one of the non-natural amino acids of an anti-CD47 antibody. Exemplary conjugation moieties and linkers have been described, e.g., see U.S. Application Publication No. US2014/0046030 A1, which is incorporated herein by reference in its entirety.

Antibodies or an antigen-binding fragments described herein that immunospecifically bind to CD47 (e.g., ECD of human CD47) can be produced by any method known in the art.

Antibodies described herein can, for example, include chimeric antibodies. A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

Antibodies or antigen-binding fragments produced using techniques such as those described herein can be isolated using standard, well known techniques. For example, antibodies or antigen-binding fragments can be suitably separated from, e.g., culture medium, ascites fluid, serum, cell lysate, synthesis reaction material or the like by conventional immunoglobulin purification procedures such as, for example, protein A-Sepharose, hydroxylapatite chromatography, gel electrophoresis, dialysis, or affinity chromatography. As used herein, an “isolated” or “purified” antibody is substantially free of cellular material or other proteins from the cell or tissue source from which the antibody is derived, or substantially free of chemical precursors or other chemicals when chemically synthesized, or from the components of the CF expression system used to produce the antibodies.

Antibodies described herein include antibody fragments which recognize specific CD47 antigens and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region. Alternatively, antibody fragments described herein can routinely be produced via well known recombinant expression techniques. See, e.g., PCT publication No. WO 92/22324; Mullinax et al., 1992, BioTechniques 12(6):864-869; Sawai et al., 1995, AJRI 34:26-34; and Better et al., 1988, Science 240:1041-1043.

Antibodies described herein can, for example, include humanized antibodies, e.g., deimmunized or composite human antibodies. A humanized antibody can comprise human constant region sequences. In certain embodiments, a humanized antibody can be selected from any class of immunoglobulins, including IgM, IgG, IgD, IgA and IgE, and any isotype, including IgG₁, IgG₂, IgG₃ and IgG₄. In certain embodiments, a humanized antibody can comprise kappa or lambda light chain constant sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s- 5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

Antibodies described herein can, for example, be multispecific, e.g., bispecific, antibodies. Methods for making multispecific (e.g, bispecific antibodies) have been described, see, for example, U.S. Pat. Nos. 7,951,917, 7,183,076, 8,,27,577, 5,837,242, 5,989,830, 5,869,620, 6,132,992, and 8,586,713.

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301.

Human antibodies can be produced using any method known in the art. For example, well known transgenic mice which are incapable of expressing functional endogenous murine immunoglobulins, but which can express human immunoglobulin genes, can be used. Alternatively, for example, phage display techniques, described above, can be utilized. Moreover, in some embodiments, human antibodies can, for example, be produced using mouse—human hybridomas. For example, human peripheral blood lymphocytes transformed with Epstein-Barr virus (EBV) can be fused with mouse myeloma cells to produce mouse—human hybridomas secreting human monoclonal antibodies, and these mouse—human hybridomas can be screened to determine ones which secrete human monoclonal antibodies that immunospecifically bind to a target antigen (e.g., ECD of human CD47). Such methods are known and are described in the art, see, e.g., Shinmoto et al., Cytotechnology, 2004, 46:19-23; Naganawa et al., Human Antibodies, 2005, 14:27-31.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

### 4.2.3 Pharmaceutical Compositions and Kits

Provided herein are compositions, pharmaceutical compositions, and kits comprising one or more protein therapeutics described herein. Also provided herein are compositions, pharmaceutical compositions, and kits comprising an anti-CD20 antibody, e.g., rituximab, alone or in combination with protein therapeutics described herein. In particular, provided herein are compositions, pharmaceutical compositions, and kits comprising one or more antibodies (e.g., anti-CD47 antibodies), described herein, or antigen-binding fragments thereof, or conjugates thereof. In certain aspects, compositions (e.g., pharmaceutical compositions) described herein can be for in vitro, in vivo, or ex vivo uses. Non-limiting examples of uses include uses to reduce immunogenicity, uses to modulate (e.g., inhibit or induce/enhance) CD47 activity, and uses to manage or treat a disorder, for example, cancer. In specific embodiments, provided herein is a pharmaceutical composition comprising an antibody (e.g., a humanized antibody) described herein (or an antigen-binding fragment thereof) and a pharmaceutically acceptable carrier or excipient.

As used herein, the term “pharmaceutically acceptable” means being approved by a regulatory agency of the Federal or a state government, or listed in the U.S. Pharmacopeia, European Pharmacopeia or other generally recognized Pharmacopeia for use in animals, and more particularly in humans.

Formulations containing one or more antibodies provided herein or an antigen-binding fragment thereof can be prepared for storage by mixing the antibody having the desired degree of purity with optional physiologically acceptable carriers, excipients or stabilizers (Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.). Such formulations can, for example, be in the form of, e.g., lyophilized formulations or aqueous solutions. Pharmaceutical carriers suitable for administration of the antibodies provided herein include any such carriers known to those skilled in the art to be suitable for the particular mode of administration. Acceptable carriers, excipients, or stabilizers are nontoxic to recipients at the dosages and concentrations employed, and include buffers such as phosphate, citrate, and other organic acids; and/or non-ionic surfactants such as TWEEN™, PLURONICS™ or polyethylene glycol (PEG).

Formulations to be used for in vivo administration can be sterile. This can be readily accomplished, for example, by filtration through, e.g., sterile filtration membranes.

In specific aspects, the pharmaceutical compositions for use in the methods provided herein contain therapeutically effective amounts of one or more of the protein therapeutics provided herein in a pharmaceutically acceptable carrier. In specific aspects, the pharmaceutical compositions for use in the methods provided herein contain therapeutically effective amounts of an anti-CD20 antibody, e.g., rituximab, alone or in combinatoin with one or more protein therapeutics provided herein, in a pharmaceutically acceptable carrier. In specific aspects, the pharmaceutical compositions for use in the methods provided herein contain therapeutically effective amounts of one or more of the antibodies or antigen-binding fragments provided herein in a pharmaceutically acceptable carrier. Such pharmaceutical compositions are useful in the prevention, treatment, management or amelioration of a condition or disorder described herein or one or more symptoms thereof.

Compositions for use in the methods provided herein can contain one or more protein therapeutics provided herein. Compositions for use in the methods provided herein can contain rituximab, alone or in combination with one or more protein therapeutics provided herein. Compositions for use in the methods provided herein can contain one or more antibodies provided herein or an antigen-binding fragment thereof. In one embodiment, compositions are provided wherein antibodies or antigen-binding fragments described herein are formulated into suitable pharmaceutical preparations, such as solutions, suspensions, powders, sustained release formulations or elixirs in sterile solutions or suspensions for parenteral administration, or as transdermal patch preparation and dry powder inhalers.

In one embodiment, compositions for use in the methods provided herein are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

In certain aspects, an anti-CD20 antibody, e.g., rituximab, is included in the pharmaceutically acceptable carrier in an effective amount sufficient to exert a therapeutically useful effect in the absence of, or with minimal or negligible, undesirable side effects on the patient treated.

In certain aspects, protein therapeutics for use in the methods provided herein are included in the pharmaceutically acceptable carrier in an effective amount sufficient to exert a therapeutically useful effect in the absence of, or with minimal or negligible, undesirable side effects on the patient treated.

In certain aspects, antibodies for use in the methods provided herein are included in the pharmaceutically acceptable carrier in an effective amount sufficient to exert a therapeutically useful effect in the absence of, or with minimal or negligible, undesirable side effects on the patient treated.

Concentrations of a protein therapeutic, for example, an anti-CD47 antibody, in a pharmaceutical composition for use in the methods provided herein will depend on, e.g., the physicochemical characteristics of the antibody, the dosage schedule, and amount administered as well as other factors.

Pharmaceutical compositions for use in the methods described herein are provided for administration to humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, such as tablets, capsules, pills, powders, granules, and oral or nasal solutions or suspensions, and oil-water emulsions containing suitable quantities of a protein therapeutic or pharmaceutically acceptable derivatives thereof. The protein therapeutic is, in one embodiment, formulated and administered in unit-dosage forms or multiple-dosage forms. Unit-dose forms as used herein refers to physically discrete units suitable for human or animal (e.g., mammal) subjects and packaged individually. Each unit-dose contains a predetermined quantity of a protein therapeutic sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles. Hence, in specific aspects, multiple dose form is a multiple of unit-doses which are not segregated in packaging.

In certain embodiments, one or more protein therapeutics for use in the methods described herein are in a liquid pharmaceutical formulation. Liquid pharmaceutically administrable compositions can, for example, be prepared by dissolving, dispersing, or otherwise mixing an antibody and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Methods of preparing such dosage forms are known, or will be apparent, to those skilled in this art; for example, see, e.g., Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.

Parenteral administration, in one embodiment, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing a protein therapuetic for use in the methods described herein is an effective mode of administration. Another embodiment is a sterile aqueous or oily solution or suspension containing a protein therapuetic for use in the methods described herein injected as necessary to produce the desired pharmacological effect.

In specific embodiments, a protein therapuetic for use in the methods described herein can be suspended in micronized or other suitable form. The form of the resulting mixture depends upon a number of factors, including the intended mode of administration and the solubility of the compound in the selected carrier or vehicle.

In other embodiments, the pharmaceutical formulations are lyophilized powders, which can be reconstituted for administration as solutions, emulsions and other mixtures. They can also be reconstituted and formulated as solids or gels.

Lyophilized powder can, for example, be prepared by dissolving a protein therapuetic for use in the methods provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. A suitable solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides an example of a formulation. In one embodiment, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain aspects, a protein therapuetic for use in the methods provided herein can be formulated for local administration or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

Anti-CD47 antibodies, anti-CD20 antibodies, e.g., rituximab, and other protein therapuetics for use in the methods provided herein can also be formulated to be targeted to a particular tissue, organ, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In some embodiments, anti-CD47 antibodies described herein are targeted (or otherwise administered) to the visual organs, bone marrow, gastrointestinal tract, lungs, brain, or joints. In specific embodiments, an anti-CD47 antibody described herein is capable of crossing the blood-brain barrier.

### 5. EXAMPLES

The examples in this section (i.e., Section 5) are offered by way of illustration, and not by way of limitation.

### 5.1 Example 1

### Co-Dosing of Rituximab and an Anti-CD47 Antibody in Cynomolgus Monkeys

The objective of the study was to monitor for immunogenicity to a humanized anti-CD47 antibody comprising the heavy chain variable region CDRs of SEQ ID NOs:50, 72, and 52 and the light chain variable region CDRs of SEQ ID NOs.: 53, 71, and 55 (hereinafter “the Anti-CD47 Antibody”) when co-dosed with rituximab or rituximab and methotrexate and evaluate its impact on pharmacokinetics following intravenous administration in cynomolgus monkeys.

In Part One of this study, the Anti-CD47 Antibody was administered as four intravenous injections (IV) to 3 groups of cynomolgus monkeys (5 animals/group, 15 animals total) on Study Days 1,8,15 and 22 to Group 1 and Study Days 15, 22, 29 and 36 to Groups 2 and 3, at doses of 20 mg/kg (Doses 1 through 4). Rituximab was administered as four intravenous injections to Group 2 and Group 3 animals, on Study Days 1, 8, 15 and 22 at doses of 10 mg/kg. Methotrexate, was administered as three subcutaneous injections to Group 3 animals, on Study Days 15, 16 and 18 at doses of 0.4 mg/kg.

In part two of this study, the Anti-CD47 Antibody was administered as a single intravenous injection to Group 1 and Group 3 animals on Study Day 78 at a dose of 20 mg/kg (Dose 5). Methotrexate was administered as multiple subcutaneous injections to Group 3 animals on Study Days 71, 72, 74, 78, 79, 81, 88, 95, 102 and 109 at a dose of 0.4 mg/kg.

Concentration data following the first, fourth and fifth dose of the Anti-CD47 Antibody in cynomolgus monkeys were used for pharmacokinetic (PK) assessments, to understand the effect of co-dosed drugs on the PK of the Anti-CD47 Antibody. Immunogenicity of the Anti-CD47 Antibody was assessed by testing for anti-drug antibody (ADA) titers in serum (with the “drug” being the Anti-CD47 Antibody), weekly throughout the study. B cell counts were followed weekly, throughout the course of the study.

All animals dosed with the Anti-CD47 Antibody alone (Group 1) turned ADA positive prior to Dose 4. The Anti-CD47 Antibody area under the serum concentration-time curve from time zero to 168 h (AUC₀₋₁₆₈) post Dose 4, a measure of the Anti-CD47 Antibody serum concentration, for the five animals of Group 1, were 3-37% of the AUC₀₋₁₆₈ post Dose 1.

This decrease in AUC post sero-conversion was attributed to ADA-mediated decrease in exposure to free Anti-CD47 Antibody. CD20+ B cell numbers in these animals were within normal range. Peripheral CD20+ B cell numbers for all animals that received rituximab (Groups 2 and 3) decreased rapidly and were <100 cells/μl within 24 hours of receiving the first dose of rituximab B cell numbers started recovering around Day 15 post-first-rituximab-dose in 8 of 10 animals in Groups 2 and 3. This recovery of B cells is attributable to anti-rituximab antibodies, as all animals had detectable anti-rituximab antibodies by Day 15, with corresponding decreases in serum rituximab concentrations. The 8 animals that showed recovery in B cell numbers had turned positive for the presence of anti drug-antibodies (ADA) by Day 36, with varying titers between 5 to 625, and varying levels of decreases in exposure in all 8 animals ranging from 2-53% of the AUC₀₋₁₆₈ post Dose 1.

In animals 11 and 15, where significant rituximab concentrations were maintained, peripheral CD20+ B cell counts remained negligible until Day 36, when the fourth dose of the Anti-CD47 Antibody was administered to these animals. These two animals, which received all 4 doses of the Anti-CD47 Antibody in the context of effective B cell depletion, remained ADA negative for prolonged time, until Day 71 and the end of the study, respectively. Anti-CD47 antibody concentrations post dose 4 were similar to the concentrations observed post dose 1, with Week 4 AUC₀₋₁₆₈ being 115% and 83% of mean week 1 AUCs in animals 11 and 15, respectively.

Monkeys treated with the Anti-CD47 Antibody alone (Group 1) or the combination of all three agents (Group 3) were subsequently re-challenged with the Anti-CD47 Antibody at 12 weeks (Day 78). In the two animals 11 and 15, where ADA development was mitigated with effective B cell depletion, PK profiles at week 12 were comparable to PK profiles post-Dose 1 of the Anti-CD47 Antibody, and AUC₀₋₁₆₈ at week 12 was 127% and 120% of mean AUC₀₋₁₆₈ post-first-dose, thus confirming that there was no ADA-mediated loss in exposure in these animals. Additionally, serum anti-drug antibody levels were sustained at low levels in these two animals, with detectable drug being present for three weeks post last dose.

Anti-CD47 Antibody concentrations in the ADA positive animals were below limit of quantitation beyond 4 days post last dose. These data indicate that B cell depletion with rituximab reduces the immune response to the Anti-CD47 Antibody and increases exposure to drug over sustained periods of time. Additionally, no toxicity in the Anti-CD47 Antibody plus rituximab or the Anti-CD47 Antibody plus rituximab plus methotrexate-treated monkeys was observed.

### 5.2 Example 2

### Clinical Study of an Anti-CD47 Antibody in Combination with Rituximab

Described herein is an open-label, Phase 1 dose escalation and expansion study of the Anti-CD47 Antibody administered by intravenous (IV) infusion, in subjects with advanced, refractory solid and hematologic cancers. The study is comprised of 2 parts. Part A is a dose escalation phase that utilizes escalating doses of the Anti-CD47 Antibody, and Part B is a dose escalation and expansion phase in which the Anti-CD47 Antibody in combination with rituximab is administered in subjects with CD20-positive non-Hodgkin's lymphoma (NHL). Expansion may occur at the maximum tolerated dose (MTD) established in the dose escalation phase and/or at a lower dose, or an alternative tolerable dosing schedule, based on review of safety, PK, and pharmacodynamics data from Part A. A modified 3+3 dose escalation design is used to identify the initial toxicity of the Anti-CD47 Antibody. Cohorts of 3 to 6 evaluable subjects are treated with the Anti-CD47 Antibody in defined dosing schedules and, in the event of dose limiting toxiticity (DLT) in one subject, cohorts are expanded to the full cohort of 6 evaluable subjects. In a given dose cohort, subjects number 4 to 6 may be enrolled before the first 3 subjects complete Cycle 1 to obtain additional safety information and to ensure sufficient number of evaluable patients for DLT assessment. No more than one subject per day is enrolled in a given dose escalation cohort.

Doses include 0.3 mg/kg IV once weekly (QW) and 1, 2, 4, 8, 15, and 20 mg/kg IV once every 2 weeks (Q2W). During dose escalation, the decision to evaluate a lower dose cohort, intermediate dose cohorts, an alternate dosing interval, or declare an MTD is determined, based on review of clinical and laboratory safety data for prior dose cohorts. All treatments are administered in 28-day cycles, with the Anti-CD47 Antibody administered on D1 and D15. After the first dose is administered in any cohort, subjects are observed for at least 28 days (Cycle 1, DLT window) before the next higher, protocol-specified dose cohort can begin.

The starting dose for Cohort 1 is 0.3 mg/kg on Day 1 of Cycle 1 followed by 1 mg/kg QW thereafter.

For Part B, in subjects with relapsed or refractory CD20-positive non-Hodgkin's lymphoma who have received prior rituximab, rituximab dosing at 375 mg/m² once a week begins two weeks prior to administration of the Anti-CD47 Antibody. Dosing of rituximab is in four weekly doses before and during Cycle 1 (D-15, D-8, D-1, D8), once every cycle for Cycles 2-6 (on D8), and, if responding, every two months thereafter for up to 12 cycles (Cycles 8, 10, and 12). Serial B cell counts and PK are assessed. In addition, PK, PD, and ADA assessments are performed as they are for part A.

### Inclusion Criteria:

1. Men and women, 18 years or older, with advanced, relapsed or refractory solid tumors, Multiple Myeloma (MM) or non-Hodgkin's lymphoma (NHL) in Part A. In Part B, relapsed or refractory CD20-positive NHL subjects only.
2. At least one site of measurable disease in subjects with solid tumors and NHL.
3. Eastern Cooperative Oncology Group Performance Status (ECOG PS) of 0 or 1.
4. Subjects must have adequate hematopoietic, liver, renal and coagulation function as assessed by specific laboratory criteria.
5. Females and males must agree to contraceptive methods and avoid conceiving throughout the study, and for up to 8 weeks following the last dose of the Anti-CD47 Antibody. If participating in Part B, females of child bearing potential should continue to use effective contraceptive methods for 12 months following treatment with rituximab

### Exclusion Criteria:

1. High grade lymphomas (Burkitts or lymphoblastic), plasma cell leukemia.
2. High grade, rapidly proliferative solid tumors (e.g., small cell lung cancer, germ cell tumors, neuroblastoma) with extensive tumor burden.
3. Symptomatic central nervous system involvement.
4. Impaired cardiac function or clinically significant cardiac disease.
5. Prior Red blood cell (RBC) transfusion <3 months prior to starting the Anti-CD47 Antibody.
6. Prior autologous stem cell transplant 3 months prior to starting the Anti-CD47 Antibody.
7. Prior allogeneic stem cell transplant with either standard or reduced intensity conditioning 6 months prior to starting the Anti-CD47 Antibody.
8. Prior systemic cancer-directed treatments or investigational modalities 5 half lives or 4 weeks prior to starting the Anti-CD47 Antibody, whichever is shorter.
9. Major surgery 2 weeks prior to starting the Anti-CD47 Antibody.
10. Pregnant or nursing females.
11. Known HIV infection.
12. Known chronic hepatitis B or C (HBV/HCV) infection.
13. Ongoing treatment with chronic, therapeutic dosing of anti-coagulants.
14. History of autoimmune hemolytic anemia or autoimmune thrombocytopenia.
15. History of concurrent second cancers requiring active, ongoing systemic treatment.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method of reducing immunogenicity in a subject, comprising administering to a subject in need thereof a therapeutically effective amount of an antibody that binds to CD47 or an antigen-binding fragment thereof, wherein the method additionally comprises administering rituximab to the subject,
wherein the antibody that binds to CD47 or antigen-binding fragment thereof comprises a VH comprising SEQ ID NO: 90 or 91, or a heavy chain of any one of SEQ ID NOs: 81-87; and a light chain comprising SEQ ID NO: 88 or SEQ ID NO: 88 without amino acid M at the N-terminus.

2-11. (canceled)

12. The method of claim 1, wherein the antibody that binds to CD47 or antigen-binding fragment thereof is an IgG isotype selected from the group consisting of IgG1 isotype, IgG2 isotype, IgG3 isotype, and IgG4 isotype.

13. The method of claim 1, wherein the antibody that binds to CD47 or antigen-binding fragment thereof is an IgG isotype selected from IgG1, IgG4P and IgG4PE.

14. The method of claim 11, wherein the antibody that binds to CD47 or antigen-binding fragment thereof is a component of a pharmaceutical composition comprising the antibody that binds to CD47 or an antigen-binding fragment thereof and a pharmaceutically acceptable carrier.

15. (canceled)

16. The method of claim 1, wherein the subject is a human.

17. The method of claim 1, further comprising administering chemotherapy.

18. The method of claim 17, wherein said chemotherapy is radiotherapy.

19. The method of claim 1, wherein the antibody that binds to CD47 or antigen-binding fragment thereof is administered to the subject at a dose of 0.3, 1, 2, 4, 8, 15, or 20 mg/kg.

20. The method of claim 1, wherein the rituximab is administered to the subject at a dose of 300, 325, 350, 375, 400, 425, 450, or 500 mg/m 2 .

21. The method of claim 1, wherein the rituximab is administered prior to the antibody that binds to CD47 or antigen-binding fragment thereof.

22. The method of claim 1, wherein the method does not comprise administering a proteosome inhibitor to the subject.

23. The method of claim 22, wherein the method does not comprise administering bortezomib to the subject.

24. The method of claim 1, wherein the method does not comprise administering methotrexate to the subject.

25-50. (canceled)
